(19) United States
(12) Patent Application Publication
(10) Pub. No.: US 2025/0245822 A1
(43) Pub. Date: Jul. 31, 2025

(54) Automated Pre-Checks To Evaluate Whether Medical Imaging Data Is Suitable For Surgical Planning Purposes

(71) Applicant: MAKO Surgical Corp., Weston, FL (US)

(72) Inventors: Casey Yusuf Chang, Davie, FL (US); Daphny Figueroa, Royal Oak, MI (US); Thomas Joseph Gibbons, Manchester (GB); Richard James Haworth, Stockport (GB); Nikolas Lessmann, Gouda (NL); Manuel Jean-Marie Urvoy, Plouzane' (FR); Thies Wuestemann, Freiburg (DE); Jason Karl Otto, Sioux Falls, SD (US)

(73) Assignee: MAKO Surgical Corp., Weston, FL (US)

(21) Appl. No.: 19/039,811

(22) Filed: Jan. 29, 2025

Related U.S. Application Data
(60) Provisional application No. 63/626,742, filed on Jan. 30, 2024.

(51) Int. Cl. G06T 7/00; A61B 34/10; G06T 7/10; G16H 30/40

(52) U.S. Cl. CPC G06T 7/0012; A61B 34/10; G06T 7/10; G16H 30/40

(57) ABSTRACT

An automated image checking suite, software program, and method to automatically evaluate whether medical imaging data of a target anatomy is acceptable to facilitate surgical planning for the target anatomy. The automated image checking suite is configured to execute one or more automated checks to determine, for example: if medical imaging data was scanned according to acceptable configuration settings; if target anatomy in the medical imaging data is acceptably captured within a boundary of the medical imaging data; if the patient moved during scanning; if a required feature of the target anatomy, which must be fully captured in the medical imaging data to acceptably facilitate surgical planning of a selected implant relative to the target anatomy, is acceptably captured within the boundary of the medical imaging data; and/or if the medical imaging data acceptably shows an intended type of target anatomy and an intended operative side of the target anatomy.

### CROSS-REFERENCE TO RELATED APPLICATIONS

The subject application claims priority to and all the benefits of U.S. Provisional Application No. 63/626,742, filed Jan. 30, 2024, the entire contents of which is hereby incorporated by reference.

### BACKGROUND

The surgical planning process for orthopedic joint replacement surgery typically begins with acquiring medical imaging data of an anatomical joint of the patient. A treatment planning request is submitted to a surgical planning team. The surgical planning team reviews patient information and manually evaluates the imaging data. A segmentation process is manually performed by a segmentation specialist to outline the bone(s) in the numerous slices of the imaging data. The output of the segmentation process is a virtual model of the bone(s). The surgical planning team or the surgeon uses the virtual model as a reference for virtually planning a type, size, and position of joint replacement implant(s) for the bone(s), as well as the proposed surgical workflow. The virtual surgical plan, including the virtual model, is then registered to the physical bone during surgery to provide intraoperative guidance to the surgeon.

There are several shortcomings with the typical review and planning process. One difficulty is that the imaging data inputted to the segmentation team may be deficient or exhibit errors. Such imaging data is therefore unsuitable for segmentation, bone model creation, or intraoperative purposes, such as visualizing the bone model on a display or registering the bone model to the physical bone. For instance, imaging data may be unsuitable for planning or intraoperative surgical purposes if the image of the bone was not acquired with the proper scanner configuration settings, fails to include the required detail of the bone, clips out a portion of the target bone, and/or is not sufficiently large for navigation visualization purposes. Another potential deficiency may be if the imaging data fails to show what is intended. For instance, the patient information may indicate that the patient requires surgical planning for the left knee, but the medical image may be of the right knee. Other errors may include the imaging data exhibiting an existing implant or metal (indicative of a revision surgery) whereas the patient information contrarily indicates a request for a primary surgery. The imaging data may also exhibit noise or blur, which would render the image quality insufficient for segmentation.

If such image errors are overlooked, the issues will cascade downstream to potentially affect accuracy and completeness of described aspects of the planning process and surgical workflow. The issues are intensified by the fact that hundreds of thousands of patients require surgical planning each year.

### SUMMARY

This Summary introduces a selection of concepts in a simplified form that are further described below in the Detailed Description below. This Summary is not intended to limit the scope of the claimed subject matter nor identify key features or essential features of the claimed subject matter.

According to a first aspect, an automated image checking suite, a non-transitory computer readable, computer program product, or computer implemented method is provided to automatically evaluate whether a medical imaging data of a target anatomy is acceptable to facilitate surgical planning for the target anatomy, by being configured to: execute automated checks to determine whether: the medical imaging data was scanned according to acceptable configuration settings; the target anatomy in the medical imaging data is acceptably captured within a boundary of the medical imaging data; the medical imaging data exhibits a motion rod that is visible above a threshold level of visibility; the medical imaging data acceptably shows an intended type of target anatomy and an intended operative side of the target anatomy; and automatically reject the medical imaging data as being unacceptable to facilitate surgical planning in response to a determination that any one or more of the automated checks produces an unacceptable result.

According to a second aspect, an automated image checking suite, a non-transitory computer readable, computer program product, or computer implemented method is provided to automatically evaluate a medical imaging data of a target anatomy, by being configured to: receive the medical imaging data as an input, wherein a type and a laterality of the target anatomy are unclassified in the medical imaging data at a time of input; automatically classify a type of the target anatomy in the medical imaging data using a first machine learning model; utilize the classified type of the target anatomy to select a second machine learning model specifically trained to classify a laterality of the classified type of target anatomy; automatically classify the laterality of the target anatomy in the medical imaging data using the second machine learning model; and produce a computer-generated output to identify the classified type and the classified laterality of the target anatomy in the medical imaging data.

According to a third aspect, an automated image checking suite, a non-transitory computer readable, computer program product, or computer implemented method is provided to automatically evaluate whether a medical imaging data of a target anatomy is acceptable to facilitate surgical planning for the target anatomy, by configured to: automatically identify and fit a shape model to the target anatomy in the medical imaging data; automatically compare a feature of the shape model to a boundary of the medical imaging data; and automatically reject the medical imaging data as being unacceptable to facilitate surgical planning in response to a determination that the feature of the shape model exceeds the boundary of the medical imaging data.

According to a fourth aspect, an automated image checking suite, a non-transitory computer readable, computer program product, or computer implemented method is provided to automatically evaluate whether a medical imaging data of a target anatomy is acceptable to facilitate surgical planning for the target anatomy, by being configured to: automatically compare the medical imaging data to a statistical population of medical imaging datum including other anatomies comparable to the target anatomy to identify an anatomical landmark of the target anatomy that is required to be visible in the medical imaging data; automatically evaluate the medical imaging data to determine whether the anatomical landmark is visible in the medical imaging data; and automatically reject the medical imaging data as being unacceptable to facilitate surgical planning in response to a determination that the anatomical landmark fails to be visible in the medical imaging data.

According to a fifth aspect, an automated image checking suite, a non-transitory computer readable, computer program product, or computer implemented method is provided to automatically evaluate whether a medical imaging data of a target anatomy is acceptable to facilitate surgical planning for the target anatomy, by being configured to: automatically obtain a measurement of the target anatomy in the medical imaging data; based on the measurement of the target anatomy in the medical imaging data, automatically select an implant from among a plurality of implant options, and automatically obtain an implant measurement of the selected implant; automatically determine, based on the implant measurement, a required feature of the target anatomy that must be fully captured in the medical imaging data to acceptably facilitate surgical planning of the selected implant relative to the target anatomy; and automatically reject the medical imaging data as being unacceptable to facilitate surgical planning in response to a determination that the required feature of the target anatomy fails to be fully captured in the medical imaging data.

According to a sixth aspect, an automated image checking suite, a non-transitory computer readable, computer program product, or computer implemented method is provided to automatically evaluate whether a medical imaging data of a target anatomy is acceptable to facilitate surgical planning for the target anatomy, by being configured to: automatically obtain a measurement of the target anatomy in the medical imaging data; based on the measurement of the target anatomy in the medical imaging data, automatically select an implant from among a plurality of implant options, and automatically obtain an implant measurement of the selected implant; automatically compare the implant measurement to a boundary of the medical imaging data; and automatically reject the medical imaging data as being unacceptable to facilitate surgical planning in response to a determination that the implant measurement exceeds a boundary of the medical imaging data or fails to be spaced from the boundary of the medical imaging data by a threshold distance.

According to a seventh aspect, an automated image checking suite, a non-transitory computer readable, computer program product, or computer implemented method is provided to automatically evaluate whether a medical imaging data of a target anatomy is acceptable to facilitate surgical planning for the target anatomy, by being configured to: obtain pre-operative patient data associated with the medical imaging data, the pre-operative patient data comprising information indicative of intended parameters comprising: (1) an intended type of target anatomy and (2) an intended operative side of the target anatomy; automatically apply the medical imaging data to a machine learning model to analyze the medical imaging data to output predicted parameters comprising: (1′) a predicted type of the target anatomy and (2′) a predicted operative side of the target anatomy; automatically compare each intended parameter to its corresponding predicted parameter; and automatically approve the medical imaging data as being acceptable to facilitate surgical planning in response to a determination that each intended parameter acceptably matches its corresponding predicted parameter.

According to an eighth aspect, an automated image checking suite, a non-transitory computer readable, computer program product, or computer implemented method is provided to automatically classify a CT volume of an anatomical joint, by being configured to: automatically generate, from the CT volume, a plurality of digitally reconstructed radiographs that are two-dimensional and that capture structures of the anatomical joint; and automatically apply each of the digitally reconstructed radiographs to a convolutional neural network that is configured to analyze each of the digitally reconstructed radiographs to output (1′) a predicted type of anatomical joint, (2′) a predicted operative side of the anatomical joint, and (3′) a predicted presence or absence of an existing implant for the anatomical joint.

According to a ninth aspect, an automated image checking suite, a non-transitory computer readable, computer program product, or computer implemented method is provided to automatically evaluate whether a medical imaging data of a target anatomy is acceptable to facilitate surgical planning for the target anatomy, by being configured to: automatically obtain one or more configuration settings defining how the medical imaging data was scanned by an imaging device; automatically compare the one or more configuration settings to one or more acceptable configuration settings; and automatically reject the medical imaging data as being unacceptable to facilitate surgical planning in response to a determination that the one or more configuration settings fail to correspond to the one or more acceptable configuration settings.

According to a tenth aspect, an automated image checking suite, a non-transitory computer readable, computer program product, or computer implemented method is provided to automatically evaluate whether a medical imaging data of a target anatomy is acceptable to facilitate surgical planning for the target anatomy, by being configured to: automatically identify a motion rod in a volume of the medical imaging data; automatically evaluate the volume of the medical imaging data to determine if the volume acceptably exhibits the motion rod; automatically reject the medical imaging data as being unacceptable to facilitate surgical planning in response to a determination that volume fails to acceptably exhibit the motion rod.

According to an eleventh aspect, an automated image checking suite, a non-transitory computer readable, computer program product, or computer implemented method is provided to automatically: identify and fit a shape model to the target anatomy in the medical imaging data; compare the shape model to a boundary of the medical imaging data; determine that a portion of the shape model exceeds the boundary of the medical imaging data; and modify the medical imaging data to capture the portion of the shape model that exceeds the boundary of the medical imaging data.

According to a twelfth aspect, an automated image checking suite, a non-transitory computer readable, computer program product, or computer implemented method is provided to automatically: obtain a measurement of the target anatomy in the medical imaging data; based on the measurement of the target anatomy in the medical imaging data, select an implant from among a plurality of implant options, and obtain an implant measurement of the selected implant; determine, based on the implant measurement, a required feature of the target anatomy that must be fully captured in the medical imaging data to acceptably facilitate surgical planning of the selected implant relative to the target anatomy; determine that the required feature of the target anatomy fails to be fully captured in the medical imaging data; identify and fit a shape model to the target anatomy in the medical imaging data, wherein a portion of the shape model exceeds a boundary of the medical imaging data; and modify the medical imaging data to capture the portion of the shape model that exceeds the boundary of the medical imaging data.

According to a thirteenth aspect, an automated image checking suite, a non-transitory computer readable, computer program product, or computer implemented method is provided to automatically: identify and fit an implant shape model to the target anatomy in the medical imaging data; compare the implant shape model to a boundary of the medical imaging data; determine that a portion of the implant shape model exceeds the boundary of the medical imaging data; and modify the medical imaging data to capture the portion of the implant shape model that exceeds the boundary of the medical imaging data.

Any of the above aspects can be utilized individually, or in combination.

Parts of certain aspects above can be utilized in combination with other parts of other aspects.

Any of the above aspects can be utilized individually, or in combination, with any one or more of the following implementations:
The feature of the shape model can include a boundary of the shape model. The boundary of the shape model can be compared to the boundary of the medical imaging data. The medical imaging data can be rejected being unacceptable to facilitate surgical planning in response to a determination that the boundary of the shape model exceeds the boundary of the medical imaging data; the feature of the shape model can include one or more points located on a boundary of the shape model. The one or more points of the shape model can be compared to the boundary of the medical imaging data. The medical imaging data can be rejected as being unacceptable to facilitate surgical planning in response to a determination that the one or more points of the shape model exceeds the boundary of the medical imaging data. The one or more points on the shape model can correspond to predetermined anatomical landmark of the target anatomy. The medical imaging data can be automatically approved as being acceptable to facilitate surgical planning in response to a determination that the feature of the shape model is within the boundary of the medical imaging data. The shape model can be derived from a statistical population of images of other anatomies that are comparable to the target anatomy. The shape model can include any one or more of: a statistical shape model, an active shape model, an active appearance model, and active contour model. An output can be automatically generated in response to rejection of the medical imaging data as being unacceptable. The output can be to generate a notification or alert to a graphical user interface to inform of rejection of the medical imaging data. The output can be to display, on a graphical user interface, the medical imaging data, and the shape model to illustrate the feature of the shape model exceeding the boundary of the medical imaging data. The output can be to generate a recommendation for a corrective action based on rejection of the medical imaging data. The medical imaging data can be a CT volume. The target anatomy can be a bone structure. Evaluating whether the medical imaging data is acceptable to facilitate surgical planning for the target anatomy can be defined as evaluating whether the medical imaging data is acceptable for later facilitating any one or more of: segmentation of the medical imaging data; virtual model creation of the target anatomy; virtual implant planning relative to the target anatomy; registration of the target anatomy to facilitate surgical navigation; and visualization of the medical imaging data to facilitate surgical navigation. The medical imaging data can be automatically approved as being acceptable to facilitate surgical planning in response to a determination that the required feature of the target anatomy is fully captured in the medical imaging data. A measurement of the target anatomy can be automatically obtained. A shape model can be automatically identified and fit to the target anatomy in the medical imaging data. The measurement of the target anatomy can be automatically obtained from the shape model. A distance between points on the shape model can be automatically measured. Each point can correspond to a predetermined anatomical landmark of the target anatomy. An implant can be automatically selected based on the measurement of the target anatomy. The implant can be automatically selected to be appropriately sized for the measurement of the target anatomy. The required feature of the target anatomy can be a required measurement and/or a required landmark. An output can be automatically generated in response to rejection of the medical imaging data as being unacceptable. The output can include to display, on a graphical user interface, the medical imaging data and the required feature of the target anatomy failed to be fully captured in the medical imaging data. The output can include to display, on a graphical user interface, or the medical imaging data and the selected implant or the implant measurement. The medical imaging data can be automatically approved as being acceptable to facilitate surgical planning in response to a determination that the implant measurement is within the boundary of the medical imaging data and/or is spaced from the boundary of the medical imaging data by the threshold distance. An implant shape model can be automatically obtained for the selected implant. A measurement of the selected implant can be obtained from the implant shape model. The implant shape model can be fit to the target anatomy in the medical imaging data. The implant shape model can include a boundary or point. The boundary or point of the implant shape model can be automatically compared to the boundary of the medical imaging data. The medical imaging data can be rejected as being unacceptable to facilitate surgical planning in response to a determination that the boundary or point of the implant shape model exceeds the boundary of the medical imaging data or fails to be spaced from the boundary of the medical imaging data by the threshold distance. The medical imaging data can be automatically rejected as being unacceptable to facilitate surgical planning in response to a determination that any one or more of the intended parameters fails to acceptably match its corresponding predicted parameter. The medical imaging data can include a CT volume. From the CT volume, a digitally reconstructed radiograph that is two-dimensional can be automatically generated. The digitally reconstructed radiograph can be automatically applied to the machine learning model to analyze the digitally reconstructed radiograph to output the predicted parameters. The machine learning model can be a convolutional neural network. The medical imaging data can be automatically applied to the convolutional neural network to analyze the medical imaging data to output the predicted parameter. Pre-operative patient data associated with the medical imaging data can be automatically obtained. The pre-operative patient data can include information indicative of intended parameters such as: an intended type of anatomical joint and an intended operative side of the anatomical joint. The medical imaging data can be automatically applied to the machine learning model to analyze the medical imaging data to output predicted parameters such as: a predicted type of anatomical joint and a predicted operative side of the anatomical joint. The medical imaging data can include a CT volume of an anatomical joint. The machine learning model can be a lightweight 2D convolutional neural network. From the CT volume, a first digitally reconstructed radiograph can be automatically generated that is two-dimensional and captures a bony structure of the anatomical joint. From the CT volume, a second digitally reconstructed radiograph can be generated that is two-dimensional and captures a soft tissue structure of the anatomical joint. The first digitally reconstructed radiograph can be automatically applied to the lightweight 2D convolutional neural network to analyze the first digitally reconstructed radiograph to output the predicted type of anatomical joint. The second digitally reconstructed radiograph can be automatically applied to the lightweight 2D convolutional neural network to analyze the second digitally reconstructed radiograph to output the predicted operative side of the anatomical joint. Pre-operative patient data can be obtained comprising information indicative of an intended parameter such as: an intended presence or absence of an existing implant for the target anatomy. The medical imaging data can be automatically applied to the machine learning model to analyze the medical imaging data to further output a predicted parameter such as a predicted presence or absence of an existing implant for the target anatomy. The intended presence or absence of an existing implant can be automatically compared with the predicted presence or absence of an existing implant. The medical imaging data can be automatically rejected as being unacceptable to facilitate surgical planning in response to a determination that the intended presence or absence of an existing implant fails to acceptably match the predicted presence or absence of an existing implant. From the CT volume, a digitally reconstructed radiograph can be automatically generated that captures a metal structure in the anatomical joint. The digitally reconstructed radiograph can be automatically applied to the convolutional neural network to analyze the digitally reconstructed radiograph to output the predicted presence or absence of an existing implant for the target anatomy. The digitally reconstructed radiograph can be automatically applied to the convolutional neural network to analyze the digitally reconstructed radiograph to output the predicted presence of the existing implant as well as one or more of: a predicted manufacturer of the existing implant; a predicted model number of the existing implant; and a predicted size or type of the existing implant. Pre-operative patient data can be obtained comprising information indicative of an intended parameter such as an intended type of procedure to be planned for the target anatomy. The medical imaging data can be automatically applied to the machine learning model to analyze the medical imaging data to further output a predicted parameter sch as a predicted type of procedure to be planned for the target anatomy. The intended type of procedure can be automatically compared with the predicted type of procedure. The medical imaging data can be automatically rejected as being unacceptable to facilitate surgical planning in response to a determination that the intended type of procedure fails to acceptably match the predicted type of procedure. The medical imaging data can be automatically approved as being acceptable to facilitate surgical planning in response to a determination that the one or more configuration settings correspond to the one or more acceptable configuration settings.

The medical imaging data can a 3D CT volume. The target anatomy can be an anatomical joint. A first machine learning model can automatically classify the type of the anatomical joint in the 3D CT volume as one of: a unilateral hip, a unilateral knee, a unilateral ankle, a bilateral hip, a bilateral knee, or a bilateral ankle. A confidence score can be generated to indicate classification accuracy of the type of the anatomical joint in the 3D CT volume. The confidence score can be compared to a threshold. In response to the confidence score exceeding the threshold, the type of anatomical joint can be automatically classified. The classified type of anatomical joint can be used to automatically select the second machine learning model. A plurality of confidence scores can be generated that indicate classification accuracy of the type of the anatomical joint in the 3D CT volume as each of: a unilateral hip, a unilateral knee, a unilateral ankle, a bilateral hip, a bilateral knee, or a bilateral ankle. A most confident score from among the plurality of confidence scores can be identified. The type of anatomical joint can be automatically classified based on the most confident score. The classified type of anatomical joint can be used to automatically select the second machine learning model. The classified type of anatomical joint can be used to automatically select the second machine learning model only in response to automatically classifying the type of the anatomical joint in the 3D CT volume as one of: a unilateral hip, a unilateral knee, or a unilateral ankle. In response to automatically classifying the type of the anatomical joint in the 3D CT volume as a unilateral hip, the second machine learning model, specifically trained to classify the laterality of the unilateral hip is a left hip or a right hip, can be selected. In response to automatically classifying the type of the anatomical joint in the 3D CT volume as a unilateral knee, the second machine learning model, specifically trained to classify the laterality of the unilateral knee is a left knee or a right knee, can be selected. In response to automatically classifying the type of the anatomical joint in the 3D CT volume as a unilateral ankle, the second machine learning model, specifically trained to classify the laterality of the unilateral ankle is a left ankle or a right ankle, can be selected. A confidence score can be generated that indicates classification accuracy of the laterality of the anatomical joint in the 3D CT volume. The confidence score can be compared to a threshold. In response to the confidence score exceeding the threshold, the laterality of the anatomical joint can be automatically classified. The classified laterality of anatomical joint can be utilized to automatically produce the computer-generated output identifying the classified laterality. A first confidence score can indicate classification accuracy of the laterality of the anatomical joint being a left-side joint. A second confidence score can indicate classification accuracy of the laterality of the anatomical joint being a right-side joint. A most confident score from among the first and second confidence scores can be identified to automatically classify the laterality of the anatomical joint based on the most confident score. A confidence score can be generated that indicates classification accuracy of the type of the target anatomy in the medical imaging data; and the medical imaging data can be automatically rejected as being unacceptable to facilitate surgical planning in response to a determination that confidence score fails to meet an acceptable threshold. A confidence score can be generated that indicates classification accuracy of the laterality of the target anatomy in the medical imaging data; and the medical imaging data can be automatically rejected as being unacceptable to facilitate surgical planning in response to a determination that confidence score fails to meet an acceptable threshold. The first and the second machine learning models can be a deep learning model comprising a convolutional neural network.

The medical image checker or automated image checking suite can be implemented in various ways, such as a software as a medical device SaMD (software intended to be used for one or more medical purposes that perform these purposes without being part of a hardware medical device), software as a service SaaS (accessing software through the internet without downloads), or the like. The imaging data may include any type of imaging data such as, but not limited to a 2D or 3D CT scan or volume, X-rays, Fluoroscopy image data, ultrasound imaging data, digitally reconstructed radiographs (DRR), or the like. The medical imaging data may be previously classified or unclassified prior to being processed using the techniques described herein.

Extrapolation of the target anatomy, shape model, implant shape model, or medical image can be utilized for any aspect. The medical imaging data can be modified to enable visualization of the shape model, including the portion of the shape model that exceeds the boundary of the medical imaging data. An extrapolation region can be generated that includes a portion of the shape model that exceeds the boundary of the medical imaging data. The extrapolation can be adjacent to the boundary of the medical imaging data or the medical imaging data can be extended to include the extrapolation region. Within the extrapolation region, features of the target anatomy within the portion of the shape model that exceeds the boundary of the medical imaging data can be artificially recreated. Soft tissue regions adjacent to the portion of the shape model that exceeds the boundary of the medical imaging data can also be artificially recreated. Color coding and/or annotations can be used to identify the portion of the shape model that exceeds the boundary of the medical imaging data.

Any of the checks described herein can be used for images exhibiting any type of anatomical structure, and the checks can be used to evaluate and/or classify any type of anatomical structure, including but not limited to: a bone; bones; soft tissue; ligaments; cartilage; osteophytes; shoulder joint bones or features, such as a glenoid, scapula or humerus, or any portions thereof; spinal bones, such as vertebral bodies, discs or cartilage, pedicles, or any portions thereof; knee joint bones, such as a femur, tibia, patella, ligaments, cartilage, or any portions thereof; hip joint bones, such as a femur, acetabulum, pelvis, cartilage, ligaments, or any portions thereof; cranial bones; facial bones; ankle bones; wrist bones; bone fragments or fractured portions. Other types of anatomies are contemplated. Additionally, any of the checks described herein can be used for images exhibiting any type of foreign body objects or existing implant and the checks can be used to evaluate and/or classified by any of the checks.

### BRIEF DESCRIPTION OF THE DRAWINGS

Advantages of the present invention will be readily appreciated as the same becomes better understood by reference to the following detailed description when considered in connection with the accompanying drawings.

FIG. 1 is a block diagram of an image processing workflow including evaluating medical imaging data with the automated image checking suite, according to one implementation.

FIG. 2 illustrates an example screenshot of a graphical user interface that can be utilized with automated image checking suite.

FIG. 3 is a method flow chart illustrating example steps of an automated image parameter pre-check, according to one implementation.

FIG. 4 is a method flow chart illustrating example steps of an automated image view pre-check, according to one implementation.

FIG. 5 is an example illustration of medical imaging data of a target anatomy wherein the automated image view pre-check evaluates a shape model or anatomical landmark with respect to a boundary of the medical imaging data.

FIG. 6 is a method flow chart illustrating example steps of an automated planning pre-check, according to one implementation.

FIG. 7 is an illustration of medical imaging data related to a target anatomy wherein the automated planning pre-check evaluates a shape model or landmark with respect to a boundary of the medical imaging data, according to one implementation.

FIG. 8 is a side view of an example implant with corresponding measurements which may be utilized by the automated planning pre-check, according to one implementation.

FIG. 9 is a method flow chart illustrating example steps of an automated image classification pre-check, according to one implementation.

FIG. 10 is a method flow chart illustrating example steps of an automated motion pre-check, according to one implementation.

FIG. 11 is a combined method flow chart and block diagram illustrating example components and steps of an automated volume classifier and laterality pre-check, according to one implementation.

FIG. 12 is a block diagram of an image processing workflow including evaluating medical imaging data with the automated image checking suite, according to another implementation.

FIG. 13 illustrates an example screenshot of a graphical user interface, which can be utilized with any aspect of the automated image checking suite, for providing an imaging checking report, according to one implementation.

FIG. 14 illustrates another example screenshot of a graphical user interface, which can be utilized with any aspect of the automated image checking suite, for providing an imaging checking report, according to another implementation.

FIG. 15 illustrates an example screenshot of a graphical user interface, which can be utilized with automated image checking suite, for providing a case file dashboard with image checking status updates, according to one implementation.

### DETAILED DESCRIPTION

Described herein are systems, computer-implemented methods, software programs, non-transitory computer readable media and/or techniques for automatically evaluating medical imaging data.

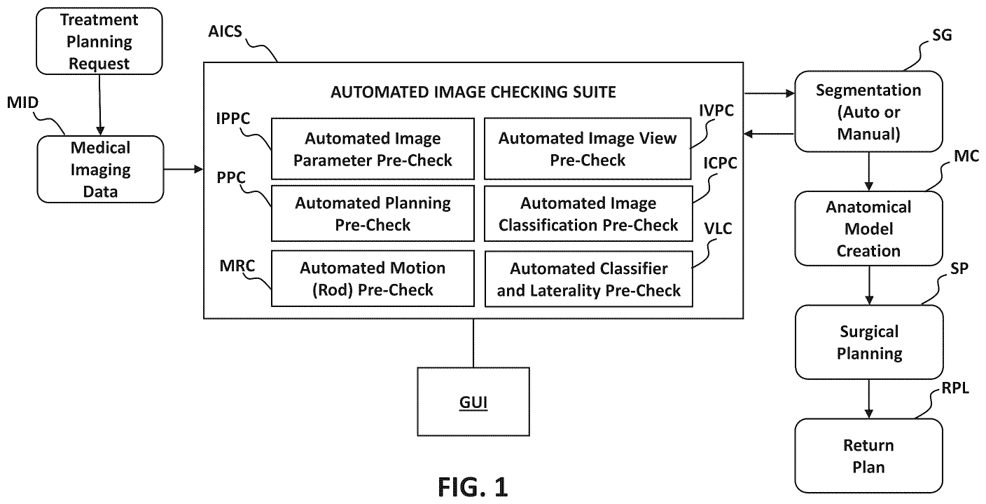

With reference to FIG. 1, an automated image checking suite AICS is provided for implementing automated evaluation of the medical imaging data MID. The term “automated image checking suite AICS” is utilized for simplicity in drafting to merely organize various aspects of the system/method/software/techniques described herein. The automated image checking suite AICS can be implemented on a local computer and/or remote server, such as a cloud computing server. Aspects or features of the automated image checking suite AICS can be implemented on any number of controllers, processors, computers, software modules, or servers. The automated image checking suite AICS can include non-transitory memory for storing instructions, which when executed by one or more processors, execute software, modules, or programs that can perform any of the aspects described herein. The processes herein can be implemented by any control system, one or more controllers, or any one or more processing devices. It is not required that any of the processes described herein be performed implicitly or explicitly by software that is designed or named as an “automated image checking suite.” For example, the software may be called a “medical image checker.”

In FIG. 1, an example workflow involving the automated image checking suite AICS is illustrated. A treatment planning request for one or more patients is generated by the hospital, organization, or surgeon. The treatment planning request can be transmitted using any suitable technique or medium. According to one example, pursuant to the treatment planning request, the automated image checking suite AICS receives medical imaging data MID for the patient. As will be described below, in another example, the treatment planning request can be created after successful processing by the automated image checking suite AICS. The medical imaging data MID may be provided simultaneously or separately from a treatment planning request. For instance, the automated image checking suite AICS can receive the imaging data MID from a PACS (picture archiving and communication system) server or any centralized computing system to enable healthcare professionals to share patient medical images and reports across various locations. The imaging data MID can be transferred to the automated image checking suite AICS using any suitable method, such as through transmission over the internet, physical connection through a data storage device, such as a flash drive, or the like. Imaging data MID can be downloaded or retrieved in bulk for many patients, or as needed on an individual patient basis.

The imaging data MID can be transferred to the automated image checking suite AICS using any suitable method, such as through transmission over the internet, physical connection through a data storage device, such as a flash drive, or the like. Imaging data MID can be downloaded or retrieved in bulk for many patients, or as needed on an individual patient basis.

The imaging data MID relates to a target anatomy TA for one or more patients. The techniques described herein apply fully to any type of target anatomy TA that may require surgical planning, such as bones, soft tissue, and the like. For instance, the target anatomy TA may be a hip joint or bone, a knee joint or bone, a shoulder joint or bone, an ankle joint or bone, a spinal vertebra, or vertebrae, a cranium, or the like. The surgical planning can be to facilitate any type of surgery, including orthopedic surgery, such as partial or total knee joint replacement surgery, partial or total hip joint replacement surgery, partial or total shoulder replacement surgery, anatomical or reverse shoulder surgery, joint fusion, arthroscopy, arthroplasty, discectomy, laminectomy, disc arthroplasty, trauma or bone fracture repair surgery, wrist repair, ankle repair, craniomaxillofacial surgery, cardiological surgery, oncological surgery, dental surgery, or the like. The surgical planning process may be used for planning the implantation of any suitable type of implant or prosthesis required by the surgery. For instance, the implant or prosthesis may be hip and knee implants, including unicompartmental, bicompartmental, or total knee implants, femoral stem implants, acetabular cup implants, orthopedic screws, pedicle screws, orthopedic plates, and the like.

The imaging data MID may include any type of imaging data such as, but not limited to a CT scan or volume, X-rays, Fluoroscopy image data, MRI scans, PET scan, ultrasound imaging data, digitally reconstructed radiographs (DRR), single-photon emission computed tomography (SPECT) image, an arthrogram, or the like. The imaging data MID can also be in any suitable file format, such as Analyze, Neuroimaging Informatics Technology Initiative (Nifti), Minc, and Digital Imaging and Communications in Medicine (DICOM). The imaging data MID can be 3D volumetric image data or any number of 2D slices.

In addition to visual imagery of the target anatomy TA, the imaging data MID may further include data or metadata. For example, data may include textual or numeral information related to the patient or to the imaging data MID. Any of the textual or numeral information may be included on the image scans themselves and/or provided in an electronic file accompanying the image scans. In one example, the imaging data MID can include data related to scanner configuration settings, such as, but not limited to: radiation dose, detector configuration (bream collimation), pixel size, resolution, window width, window level, number of slices, slice thickness, image density, intensity, automatic exposure control (AEC) parameters such as noise index or mA level during tube current modulation, signal-to-noise ratio, tube potential kV, gantry rotation, patient positioning, scan range, slice thickness and pitch, scan time, etc. The imaging data MID can also include (e.g., preoperative) patient specific or surgical information such as, but not limited to: patient name, date of birth, gender, the planned operative side of the patient, data related to the treatment request, surgeon name, type of surgery to be performed, type of planned implant, planned date of surgery, and the like.

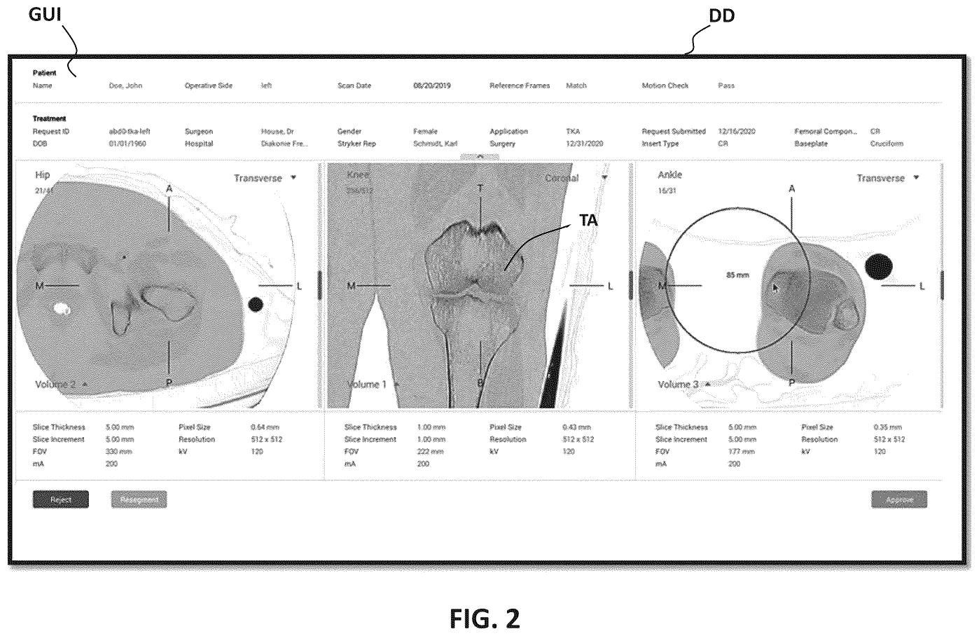

As shown in FIGS. 1 and 2, the automated image checking suite AICS can optionally be implemented with visualization, or a graphical user interface GUI that can be provided on any suitable display device DD (FIG. 2). The GUI can be a data review screen to display any suitable information related to the imaging data, such as, but not limited to: the imaging data MID from various planes or slices, slice thickness, slice increment, the field of view of the displayed imaging data, parameters of the scan (such as pixel size, resolution, voltage, current, date of the scan), patient information (such as name, date of birth, gender, etc.), the planned operative side of the patient, data related to the treatment request, surgeon name, type of surgery to be performed, type of planned implant, planned date of surgery, and the like. The GUI can be utilized to optionally enable a technician to: review the data described above, review the output of automated functionality implemented by the automated image checking suite AICS, visualize the output of segmentation or the anatomical model, and/or perform supplemental manual review (before or after automated functionality implemented by the automated image checking suite AICS).

### A. Image Pre-Checks

With reference to FIG. 1, the automated image checking suite AICS can perform one or more automated image pre-checks with respect to the imaging data MID. As used herein term “pre-check” defines an initial check performed on the received imaging data MID prior to further processing for any downstream surgical planning (e.g., segmentation, anatomical model creation, implant planning) or aspects of surgical workflow which rely on surgical planning or medical imaging (e.g., surgical navigation visualization, anatomical registration, etc.). To this end, the automated image checking suite AICS is configured to evaluate medical imaging data preoperatively, i.e., prior to the virtual surgical planning process and prior to surgery.

As will be described the several sections below, the automated image checking suite AICS is configured to perform any one or more of the following automated pre-checks: an automated image parameter pre-check IPPC, an automated image view pre-check IVPC, an automated planning pre-check PPC, and an automated image classification pre-check ICPC, an automated motion pre-check MRC, and an automated volume classifier and laterality pre-check VLC. The automated image checking suite AICS can execute any of these automated image pre-checks in several ways. In one example, any one or more of the automated pre-checks are executed individually or independently of the others. Alternatively, the automated pre-checks can be executed simultaneously, collectively, or together. Additionally, the automated image checking suite AICS can execute partial aspects of any of the pre-checks described herein or combine those partial aspects with features of other pre-checks. Furthermore, any two or more of these automated pre-checks may be combined into a single checking operation. Additionally, any two or more of these automated pre-checks may be performed in a prioritized order or arbitrary order. In some instances, the automated image checking suite AICS can selectively determine which of the automated image pre-checks to perform or not perform. For instance, the automated image checking suite AICS can make this determination in response to automatically identifying that certain information about the patient is missing from the imaging data MID. Priority of the check may be pre-defined based on the statistical or predicted likelihood of such check identifying an error. The automated image checking suite AICS can output a single report with information about the outcome of any check or a combined report with information about the outcome of the multiple checks. Moreover, described herein are techniques for extrapolating medical images and/or target anatomies beyond the original boundary of the medical image, e.g., to provide a means to salvage scans that would otherwise fail the respective check for failing to capture the requisite amount of the target anatomy.

Using these image pre-checks, the automated image checking suite AICS can automatically, rapidly, and accurately identify deficiencies or errors in the imaging data MID. In doing so, the automated image checking suite AICS can automatically determine whether or not the imaging data MID is suitable for downstream surgical planning (e.g., segmentation, anatomical model creation, implant planning) or suitable for aspects of surgical workflow which rely on surgical planning or medical imaging (e.g., surgical navigation visualization, anatomical registration, etc.). The automated image checking suite AICS can process the described pre-checks within seconds, thereby alleviating the time-consuming burden of having a surgical planning team manually review the imaging data MID to identify potential errors. In turn, the automated image checking suite AICS can substantially reduce the labor cost and human error involved with manual review of the imaging data.

### 1. Automated Image Parameter Pre-Check

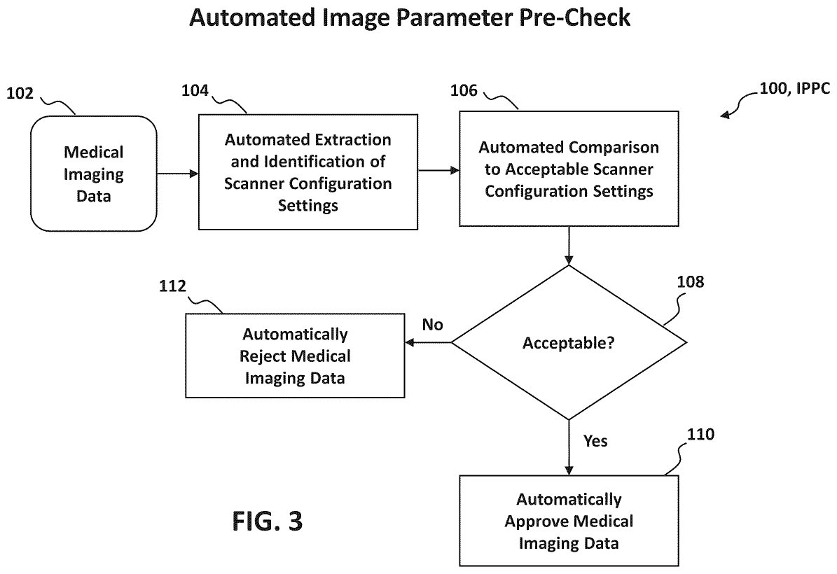

With reference to FIG. 3, the automated image checking suite AICS can perform an automated image parameter pre-check IPPC, i.e., a pre-check to determine whether the imaging data MID was acquired with acceptable image parameters or scanner configuration settings. The acceptable image parameters can be those deemed satisfactory by the vendor of the automated image checking suite AICS or acceptable according to industry standards. The settings can include, but are not limited to: radiation dose, detector configuration (bream collimation), pixel size, resolution, window width, window level, image density, intensity, automatic exposure control (AEC) parameters such as noise index or mA level during tube current modulation, signal-to-noise ratio, tube potential kV, gantry rotation, patient positioning, scan range, slice thickness and pitch, scan time, etc. The automated image checking suite AICS can include a list of values or ranges for the acceptable image settings. These values can be stored in a look-up table, for example. An example method 100 of performing the image parameter pre-check IPPC is shown in FIG. 3 and includes step 102 of receiving the imaging data MID of the target anatomy TA. The received imaging data MID include the image parameters, e.g., which may be encoded in a DICOM file. For this check, the received imaging data MID may or may not include the actual image scans of the patient. At step 104, the automated image checking suite AICS can automatically identify and extract the image parameters from the received imaging data MID. This process may include text or string search or matching algorithms, or the like. At step 106, the automated image checking suite AICS can automatically compare the extracted image parameters to the stored acceptable image parameter values.

At step 108, the comparison may be to determine whether or not the extracted values are an acceptable value or fall within a predetermined range or threshold of values. If the extracted values are acceptable, the automated image parameter pre-check IPPC, at step 110, can automatically produce a response to confirm the acceptability. In one example, the response is to send a confirmation to the GUI to enable the technician to view that this pre-check has passed or that the settings are correct. In another example, the response is for the automated image checking suite AICS to continue processing additional pre-checks, e.g., in an ordered series of checks. Other responses are contemplated, such as producing no response unless an error was detected.

If the extracted values are unacceptable, the automated image parameter pre-check IPPC, at step 112, can automatically produce a response regarding the unacceptability. In one example, the response is to send an alert or notification to the GUI to enable the technician to view that this pre-check has failed or to identify which setting(s) is/are incorrect. In another example, the response is for the automated image checking suite AICS to stop processing additional pre-checks, e.g., in an ordered series of checks. The response can also be to output a message requesting a new scan with the appropriate parameters.

### 2. Automated Image View Pre-Check

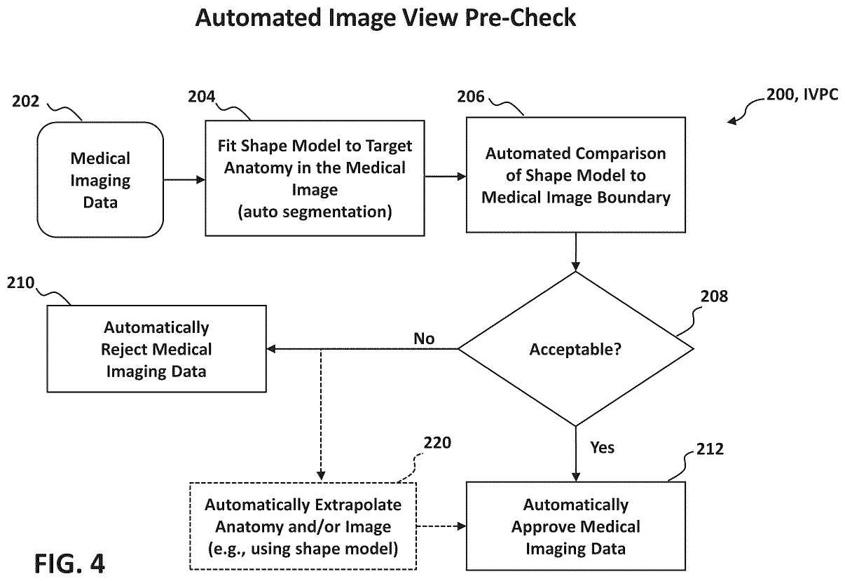

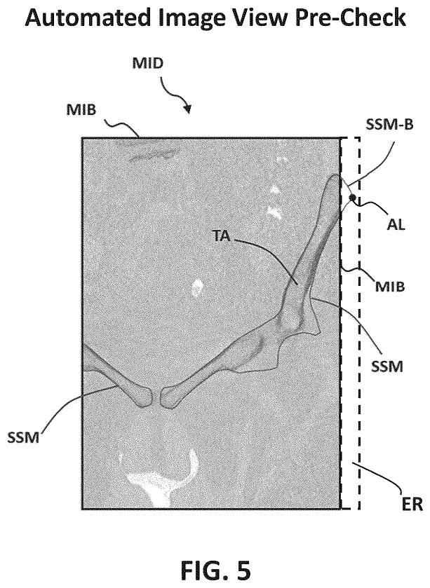

With reference to FIGS. 4 and 5, the automated image checking suite AICS can perform an image view pre-check IVPC. The image view pre-check IVPC can automatically determine: whether an image from the imaging data MID includes the required detail of the target anatomy TA (e.g., bone); whether image is not large enough; whether the target anatomy TA is adequately captured within the image view; whether the target anatomy TA is clipped; and/or whether any required portion of the target anatomy exceeds a boundary of the image MID. For each patient, the image view pre-check IVPC can be repeated for any number of slices or all slices of the medical imaging data MID which include the target anatomy TA of the patient.

The image view pre-check IVPC may be relevant for surgical planning purposes, such as segmentation or anatomical model creation purposes whereby it is important to ensure the required amount of the target anatomy is captured within the image data. Otherwise, the segmentation or anatomical model may include be incomplete or contain errors. The image view pre-check IVPC may also be relevant for navigation purposes. For instance, ensuring the required amount of target anatomy is captured within the image data ensures accuracy in visualization of the image data for surgeon reference during navigated surgery and accuracy in the anatomical registration process wherein the anatomical model is registered to the physical target anatomy TA.

An example method 200 of performing the automated image view pre-check IVPC is shown in FIG. 4 and includes step 202 of receiving the imaging data MID of the target anatomy TA. At step 204, the automated image checking suite AICS automatically identifies and fits a shape model SSM to target anatomy TA in the medical imaging data MID. The term “shape model” is used herein to include any one or more of: a statistical shape model, an active shape model, an active appearance model, an active contour model, or any other suitable type of shape model. In one example, the shape model SSM can be understood as a mesh, shape or contour that has adjustable nodes to deform the mesh, shape, or contour to substantially confirm to the shape or contour of the target anatomy TA in the medical imaging data MID. The shape model can be initially derived or generated from a population of other patient images exhibiting similar anatomies as the target anatomy TA. The population can exhibit similar characteristics of the subject patient, such as age, gender, ethnicity, size, or other physical or demographical data. For instance, when the target anatomy TA is a bone, the shape model SSM may be derived from a statistical representation of images of bones of comparable anatomical origin from a group of patients known to have normal or “healthy” bone anatomy. The shape data used to derive the shape model SSM may include geometric characteristics of a bone such as landmarks AL, surfaces, boundaries, geometric characteristics, or intensity information of a target anatomy TA. The shape data can be provided from analysis of other patient images and/or from point clouds acquired from normal bones of comparable anatomical origin. The automated image checking suite AICS can select one shape model from among a plurality of shape models that is best fit to the target anatomy. The best-fit shape model may or may not need to be morphed to the target anatomy. Alternatively, the automated image checking suite AICS can utilize one generic shape model (for the specific anatomy type) and morph it to the target anatomy. In any implementation described herein, the shape model may be a singular shape model or may be realized as a plurality of shape model instances.

The shape model SSM can be utilized by using an algorithm that automatically segments the medical imaging data MID. In one implementation, the SSM may represent “healthy” anatomy that may not necessarily correspond exactly to the target anatomy of the subject patient. The segmentation algorithm may perform image processing such as alignment of coordinates of the target anatomy TA in image data to the SSM. The alignment may be based on key marker points on the target anatomy TA in image data and the SSM. The segmentation algorithm may then morph, deform, or scale the SSM until the SSM and the target anatomy TA in the image data register. This fitting process can be performed using any optimization technique, such as a least squares optimizer. The registration may include adjusting the size and shape of the SSM to the target anatomy TA in image data and adjust a location of the SSM to align with the target anatomy TA in image data. The result of the registration may be a shape model that approximates the target anatomy TA in image data (e.g., optionally with or without osteophytes or abnormal morphology).

In another implementation, the automated segmentation algorithm performs an initial segmentation process on the image data associated with the target anatomy TA with a first shape model to generate an initial segmentation of the target anatomy TA. The automated segmentation can optionally further perform a refined segmentation process on the image region of the image data associated with the target anatomy TA using a neural network that takes as an input the image data of the target anatomy TA and the output of the initial segmentation. The first shape model can be mapped to an output of the refined segmentation process. Optionally, anatomical landmarks AL of the target anatomy TA can be identified. Examples of such automated segmentation may be implemented in a manner as described in U.S. Provisional Patent App. No. 63/505,466, filed Jun. 1, 2023, and entitled “Segmentation of Bony Structures” (Attorney Docket No. 060939.01029), the entire contents of which are hereby incorporated by reference.

As shown in FIG. 5, the shape model SSM (derived from any techniques described above) is represented as a shape that approximates the outline of the target anatomy TA (e.g., knee joint bone(s)) relative to the medical imaging data MID. The process of visualizing the shape model SSM relative to the image is optional and not necessary for this automated pre-check. If useful, a technician may manually retrieve and review any shape model relative to any corresponding image on the GUI.

In FIG. 5, and as shown at step 206 in FIG. 4, the automated image view pre-check IVPC performs an automated comparison between the shape model SSM and a boundary MIB of the medical imaging data MID. Parameters of the boundary MIB of the medical imaging data MID can be derived from the scanner configuration settings, or image parameters, and/or by automated measuring of the boundary MIB size. Most often, but not always, the boundary MIB of the medical imaging data MID will be rectangular.

In one implementation, the comparison is a boundary-to-boundary comparison, i.e., a comparison between a boundary SSM-B of the shape model SSM and the boundary MIB of the medical imaging data MID. The shape model boundary SSM-B can be determined from the parameters or size of the shape model SSM and/or by automated measuring of the boundary SSM-B. In some instances, the shape model SSM and the medical imaging data MID are registered to and compared in a common coordinate system, which may be the coordinate system or the image, the shape model, or any arbitrary coordinate system. The coordinate system in which these boundaries are measured may be larger than the boundary MIB of the medical imaging data MID to enable detection the shape model SSM beyond the image boundary MIB. The boundary comparison can be performed relative to all or some boundaries of the shape model SSM. Any aspect of this comparison may be a back end (not visualized process) and not necessarily visualized to a user.

In another implementation, instead of a boundary-to-boundary evaluation, the automated image view pre-check IVPC performs a point-to-boundary comparison. For instance, one or more anatomical landmarks AL of the target anatomy TA that were identified during the auto-segmentation process can be mapped to the shape model SSM (as shown in FIG. 5). The automated image view pre-check IVPC evaluates whether the landmark AL falls within or exceeds the image boundary MIB. In one example, the landmarks AL may be derived from clinical data that identifies what landmarks need to be visible to be able to plan and execute the surgery. In another example, the landmarks AL may be derived from those points required to perform anatomical registration during intra-operative navigated surgery. Additionally, or alternatively, the anatomical landmarks AL can be one or more points chosen automatically based on the likelihood of the landmark AL exceeding the image boundary MIB. For instance, the landmark AL can be the most anterior, posterior, medial, lateral, superior, or inferior point of a target anatomy TA structure. Alternatively, instead of a shape model, the automated image view pre-check IVPC may compare the medical imaging data MID to a statistical population of medical images including other anatomies comparable to the target anatomy to identify the anatomical landmark AL of the target anatomy that is required to be visible in the medical imaging data MID. The automated image view pre-check IVPC can automatically evaluate the medical imaging data MID to determine whether the required anatomical landmark AL is visible in the medical imaging data MID.

A point-to-point evaluation is also contemplated. The shape of the SSM may be interpolated using a plurality of points that have coordinates in the coordinate system. The medical imaging data MID may be interpolated as a grid of points or pixels. The automated image view pre-check IVPC may inspect whether the coordinates of the points of the SSM correspond or overlap to the coordinates of the points or pixels of the medical imaging data MID. The automated image view pre-check IVPC can combine any of the described techniques for comparing the SSM to the medical imaging data MID.

At step 208, the boundary comparison yields a determination of whether or not the medical imaging data MID is acceptable. In the example of FIG. 5, the automated image view pre-check IVPC identifies that an outer right contour of the shape model boundary SSM-B extends beyond the image boundary MIB. Hence, the automated image view pre-check IVPC detects one or more of the following: the image fails to include the required detail of the target anatomy TA; the image is not large enough; the target anatomy TA is not fully captured within the image view; the target anatomy TA is clipped; and/or a required portion of the target anatomy exceeds a boundary of the image MID.

If the medical imaging data MID is determined to be unacceptable as a result of this comparison, the automated image checking suite AICS, at step 210, can automatically produce a response to regarding the unacceptability. In one example, the response is to send an alert or notification to the GUI to enable the technician to view that this pre-check has failed or to identify which image slices have failed, or optionally display in the GUI the respective slice and SSM exhibiting the failed boundary comparison. The response can also be to output a message requesting that the patient needs to obtain a larger scan. In another example, the response is for the automated image checking suite AICS to stop processing additional pre-checks, e.g., in an ordered series of checks.

On the other hand, the medical imaging data MID may be determined to be acceptable as a result of this comparison. For instance, in the example of FIG. 5, if the outer right contour of the shape model boundary SSM-B were to be (hypothetically) captured inside the image boundary MIB, then the automated image view pre-check IVPC detects one or more of the following: the image successfully includes the required detail of the target anatomy TA; the image is large enough; the target anatomy TA is fully captured within the image view; the target anatomy TA is not clipped; and/or a required portion of the target anatomy falls within the boundary of the image MID. If acceptable, the automated image checking suite AICS, at step 212, can automatically produce a response to confirm the acceptability. In one example, the response is to send a confirmation to the GUI to enable the technician to view that this pre-check has passed. In another example, the response is for the automated image checking suite AICS to continue processing additional pre-checks, e.g., in an ordered series of checks. Other responses are contemplated, such as producing no response unless an error was detected.

### 2A. Anatomy/Image Extrapolation

With reference to FIGS. 4 and 5, the automated image checking suite AICS and/or image view pre-check IVPC may be equipped with the capability to predictively extrapolate or extend the target anatomy TA and/or the image MID. For example, if it is determined that a required amount of target anatomy TA is not captured in the image MID, the automated image checking suite AICS may artificially extend the target anatomy TA beyond the original boundary MIB of the image MID. Whereas above, the shape model SSM was used to evaluate the required length of the target anatomy TA in the image MID, here, the same shape model SSM or a different shape model can be further used to extend the anatomy to the length or size required to be captured.

To implement this capability, the automated image checking suite AICS automatically identifies and fits a shape model SSM to target anatomy TA in the medical imaging data MID (step 204 of FIG. 4). In FIG. 5, and as shown at step 206 in FIG. 4, an automated comparison is performed between the shape model SSM and a boundary MIB of the image MID. At 208, the comparison determines that the shape model SSM (or a portion thereof) extends beyond the boundary MIB (and would otherwise be unacceptable). For example, in the example of FIG. 5, the check identifies that an outer right contour portion of the shape model boundary SSM-B extends beyond the image boundary MIB. However, instead of rejecting the image MID at step 210, the image MID can be preserved using the extrapolation technique, thereby avoiding the need for rescanning the image MID. Namely, at step 220, the image MID can be automatically annotated, modified, reproduced or regenerated to include information about the shape model SSM, and particularly, the portion of the shape model that extends beyond the image boundary MIB. This extrapolation can be performed in a manner that allows the image MID to be automatically approved (at 212). Again, the shape model SSM used to check the boundary limits (at steps 204, 206, 208) may be the same as, or different from, the shape model SSM used for extrapolation (at step 220).

To capture the portion of the shape model that extends beyond the image boundary MIB, the automated image checking suite AICS may generate or identify an extrapolation region (ER), as shown in FIG. 5. In one example, the extrapolation region (ER) may be an artificial extension of the image MID. The extrapolation region (ER) may modify the dimensions of the boundary MIB or may be identified as a separate region from the original image MID. Inside the extrapolation region (ER), the shape model SSM portion may be visually preserved to emulate the requisite amount of target anatomy TA. When the image is later retrieved for planning purposes, the image MID can show the border SSM-B of the shape model SSM extending beyond the original border MIB of the image data MID, and optionally, within the extrapolation region (ER), for example.

In some cases, the target anatomy TA within the extrapolation region (ER) may be automatically populated with features of the anatomy TA derived from the shape model SMM or other statistical data. For example, rather than just showing a border SSM-B of the shape model SSM in the extrapolation region (ER), the region within the border SSM-B may be artificially filled in with anatomical features, such as bony features that would have been captured had the image been larger when initially scanned. Similarly, in the extrapolation region (ER), the tissue surrounding the target anatomy TA can be artificially filled in with anatomical features, such as surrounding soft tissue that would have been captured had the image been larger when initially scanned. In some cases, tissue or bony regions within the extrapolation region (ER) can be artificially filled in using scan data from adjacent slices that may include the missing features.

Additionally, or alternatively, the entire image MID may be artificially reproduced or regenerated to include extrapolation region (ER). With this technique, there would be no visible border between boundary MIB of the image MID and the extrapolation region (ER). Instead, these two regions would be seamlessly merged into one image MID. This would allow the image MID to appear as though it were originally scanned to include the requisite amount of the target anatomy TA.

The extrapolation region (ER) or features found within the extrapolation region (ER) can be distinguished using any textual or visual indicator. For example, the shape model SSM portion can be annotated, labeled, or providing with a message identifying that this portion is extrapolated, predicted or otherwise not present in the originally scanned image MID. Visually, the shape model SSM portion in the extrapolation region (ER) can be color coded, e.g., differently from the remaining part of the image or shape model SMM. Such identifications can be used to communicate to a reviewer that the target anatomy TA in the region (ER) are extrapolated and may not perfectly represent the actual scanned anatomy. The farther the extrapolation is from the original image boundary MIB, the greater the likelihood for prediction error. As such, the image MID may also include a graduated level of textual or visual indicators that denote the increased likelihood of error the further away from the original image boundary MIB. For example, in the extrapolation region (ER), a gradient color scheme can be used to highlight portions of the target anatomy nearest to the original image boundary MIB using a green color, and the green color can transition into orange and eventually a red color to highlight portions of the target anatomy that are furthest the original image boundary MIB.

The described extrapolation technique can provide many benefits for surgical procedures where medical image data often does not include the requisite amount of the target anatomy TA. For example, for revision total knee arthroplasty (TKA), planning for stems longer than 50 mm requires a longer CT scan, but there will likely be variability in actual CT scan length. The described extrapolation technique can take current CT scan images and extrapolate the required additional length based on the shape model SM. For TKA revision cases, the stems can be 50 mm, 100 mm, or 150 mm in length. To plan for stems 100 mm and 150 mm, the bones require a scan length of 150 mm and 200 mm, respectively. To avoid having 2 different CT scan protocol lengths, and to avoid a probable shorter-than-needed scan length, the described techniques may be used to extrapolate to 100 mm for primary and to 200 mm for revision. In this way, no CT would need to be rejected due to scan length, and the patient would not need to receive another CT scan.

This is also beneficial for short CT scans even for primary cases, such that CT scans will not need to be rejected based on length. For TKA primary cases, the CT scan protocol states that the scan should be ±100 mm from the joint line. Often, the scans are much shorter than that. There are minimum length requirements necessary to generate surfaces to map bone registration points and to make notching calculations for the anterior cut. The minimum scan length is dependent on implant size, but implant size cannot be estimated until the scan is segmented. Sometimes a scan must be rejected due to violating the minimum scan length. If rejected, the notice usually occurs more than 1 day after the scan. This then requires the patient to return to the CT scan facility to get another scan. To prevent rejection, the shape model SM can be trained to extrapolate the femur/tibia bone shaft to exactly ±100 mm, thereby avoiding image rejection and the need for a re-scan.

### 3. Automated Planning Pre-Check

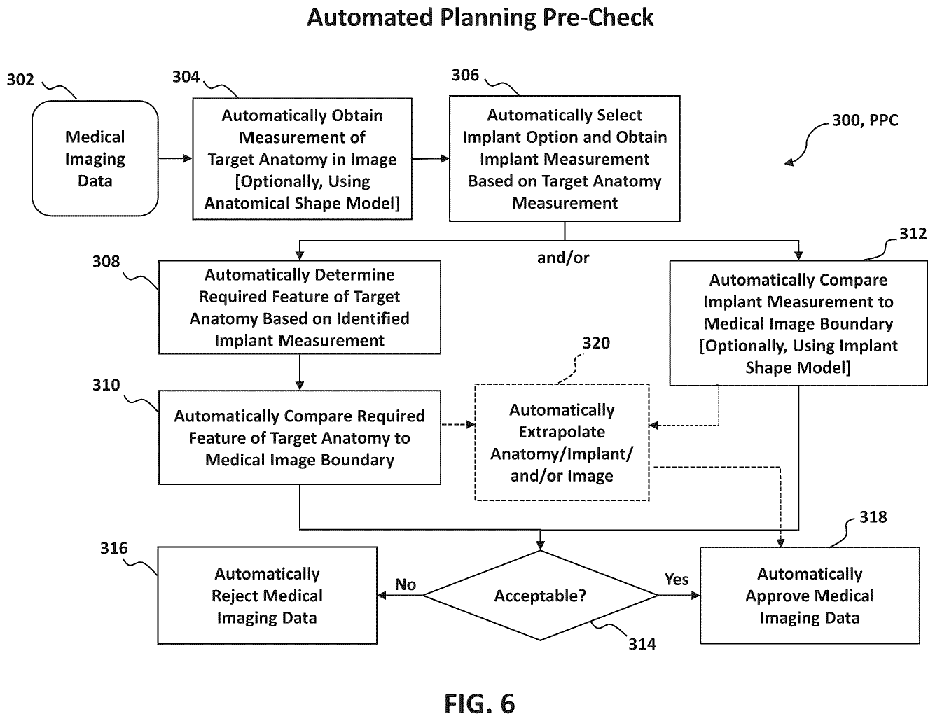

With reference to FIGS. 6-8, the automated image checking suite AICS can perform an automated planning pre-check PPC. A primary purpose of the planning pre-check PPC is to assess whether the medical imaging data MID is suitable for downstream surgical planning purposes related to an implant for the target anatomy TA. The implant used in this check is not natively found in the imaging data MID but rather is compared to the image data MID for evaluation. The implant may be a planned implant, proposed implant, best-guess implant, or theoretical implant. The planning pre-check PPC could utilize data from a surgeon's plan but is not intended to be a substitute for the surgeon's plan. Instead, the planning pre-check PPC pulls ahead data related to an implant for assessing whether the medical imaging data MID adequately shows the extent of the target anatomy TA required for implant planning purposes.

Accordingly, the planning pre-check PPC can automatically determine: whether an image from the imaging data MID includes the required detail of target anatomy TA (e.g., bone) necessary to plan an implant; whether image is not large enough to capture the required portion of the target anatomy TA given the implant; whether the target anatomy portion for which an implant will be located would not be captured within the image view; whether the target anatomy portion for which an implant will be located would be clipped; and/or whether any required portion of the target anatomy which is required for implant planning exceeds a boundary of the image MID. For each patient, the planning pre-check PPC can be repeated for any number of slices or all slices of the medical imaging data MID which include the target anatomy TA of the patient. The planning pre-check PPC could be combined with the image view pre-check IVPC, in a single check. Additionally, the planning pre-check PPC can be performed simultaneously on numerous target anatomies TA in the medical imaging data MID (e.g., such as opposing bones of an anatomical joint).

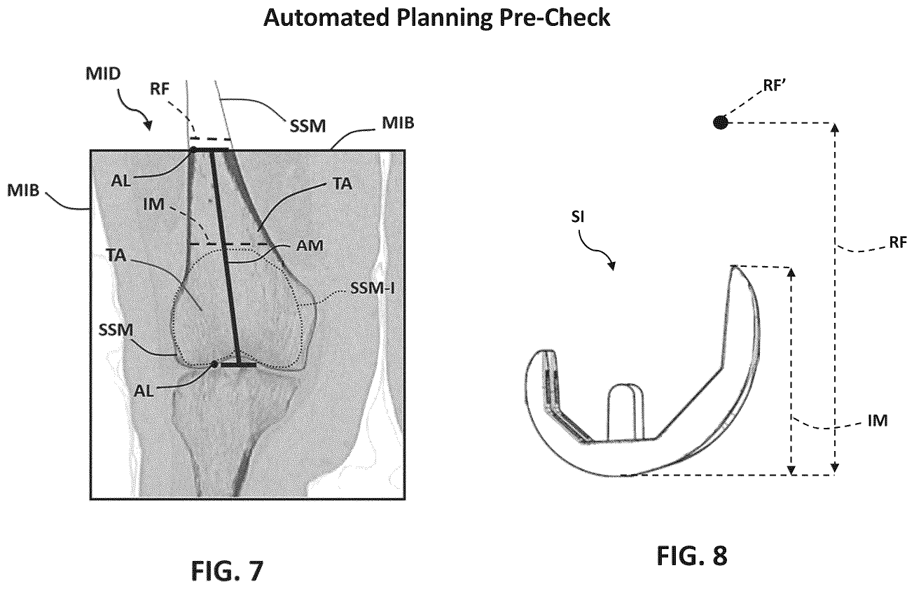

An example method 300 of performing the automated planning pre-check PPC is shown in FIG. 6 and includes step 302 of receiving the imaging data MID of the target anatomy TA. At step 304, the automated image checking suite AICS automatically performs a measurement AM of the target anatomy TA found within the image MID. In one example, this can be done by identifying landmarks AL of the target anatomy TA and measuring between the landmarks AL. For example, as shown in FIG. 7, when the image is of a knee joint, the landmarks AL can be of a distal feature of the femur, such as a condyle surface and a proximal-most point of the femur bone at the boundary MIB. This measurement can be representative of length of the femur shaft visible in the medical imaging data MID. Of course, depending on the image, target anatomy TA type, or programmed preferences, the landmarks AL may be any appropriate points or surfaces on the target anatomy TA. The automated image checking suite AICS can use a digital ruler to measure the distance between the landmarks AL. Any appropriate number of landmarks AL may be utilized.

Additionally, or alternatively, at step 304, the automated image checking suite AICS automatically obtains the target anatomy measurement AM by identifying and fitting the shape model SSM to target anatomy TA in the medical imaging data MID. The shape model SSM can be identified, generated, and/or fitted using any of the techniques described above with respect to method 200. Hence, the specific details and various implementation of the shape model SSM are fully incorporated in this section and are not repeated merely for simplicity in writing. In the example of FIG. 7, the shape model SSM is fitted to the target anatomy TA, which is a femur. As described above, the shape model SSM may extend beyond the boundary of the image MID. The parameters of the boundary of the shape model SSM include the measurements of the shape model SSM. The automated image checking suite AICS can automatically obtain the target anatomy measurement AM based on the SSM parameters and comparing such parameters to the medical image boundary MIB.

Whether landmarks AL and/or shape models SSM are utilized, the measurement of the target anatomy TA can include any one or more measurement(s), such as length, width, area, volume, perimeter, height, depth, thickness, orientation, position, varus, valgus, version, retroversion, inclination, and the like. In FIG. 7, the measurement AM recorded is the length of the femur from a condyle surface to the boundary of the image MIB, which in this example is 87 mm and is partially representative of the femur shaft length.

The method 300 includes the step of 306 identifying an implant, or implant measurements SI, based on the target anatomy measurement AM acquired at step 304. Again, the implant or implant measurements SI used in this step may be a planned implant, proposed implant, best-guess implant, or theoretical implant. In one example, the automated image checking suite AICS can automatically select, from a database, the implant or implant measurements SI having parameters appropriately sized to the target anatomy TA based on the target anatomy measurement AM. The implant or implant measurements SI selected from the database can be represented as a virtual model or SSM of the implant. Alternatively, or additionally, the selected implant or implant measurements SI may include measurement data without any graphical or virtual elements. Based on the target anatomy measurement AM, the automated image checking suite AICS can choose an implant with an appropriate size, orientation, biometric or mechanical fit, type, or configuration. The implant or implant measurements can be selected based on manufacturer, model, size identifier and/or based on anatomical measurements.

According to a first technique, at step 308, the automated image checking suite AICS can determine parameters of a required feature RF of the target anatomy based on the identified implant or implant measurements SI (from step 306). In one example, the required feature RF defines a required amount, measurement, extent, or landmark(s) of the target anatomy that must be visible in the medical imaging data MID based on the selected implant SI. For instance, if the implant selected is a size 6 femoral knee component, the implant can include data defining the geometrical measurements of the implant (such as an AP dimension, ML dimension, overhang length, etc.). The selected implant SI may also require the femur include a required shaft length to properly accommodate (in image space) the selected femoral component. For instance, as shown in FIG. 8, an example selected implant SI is shown from the sagittal view merely for illustrative purposes. The selected implant SI can be represented using numeral or textual data and need not be displayed. In this example, the selected implant SI is a femoral component of a knee prosthesis derived from the target anatomy measurement AM. The selected implant SI includes an implant measurement IM, such as a total length of the implant, e.g., from the proximal tip of the anterior flange to a distal-most point of the condyle contact surface. In one example, based on the selected size of the implant, the illustrated implant measurement IM height may be 72 mm. Of course, any other measurement may be obtained as needed.

The implant measurements retrieved from the database for this selected implant SI can include the 72 mm implant dimension as well as parameters of the required feature RF of the target anatomy that is needed for this implant. Parameters of the required feature RF for the target anatomy can be obtained from various sources, such as from the manufacturer of the implant, a threshold minimal measurement (e.g., 20% greater than the implant measurement), an implant coverage threshold, a safety tolerance, clinical or statistical data, surgeon preferences, or the like. In the example shown in FIG. 8, it may be that the required feature RF of the target anatomy (femur) is a measurement of 92 mm, which may be understood as a measurement including required femur shaft length necessary to accommodate the selected implant SI. Of course, depending on the type of target anatomy, the type of planned procedure, the specific implant measurements selected, and the specific tolerances of required anatomy, this step 308 may produce different outcomes. Alternatively, as shown in FIG. 8, the required feature can be a point or landmark RF′ defined at a terminal end of the required measurement or defined based on a pre-defined point on the femur shaft.

Continuing with the first technique, at step 310, the automated image checking suite AICS can automatically compare the required feature RF (obtained from step 308) with the anatomical measurement AM and/or medical image boundary MIB. In one example, this step can be performed by augmenting the target anatomy measurement AM originally obtained at step 304. Additionally, or alternatively, step 310 may include re-measuring the target anatomy TA in the image MID based on the required feature RF. For instance, the automated image checking suite AICS can automatically register or correlate the reference coordinates from where the implant measurements are taken to the corresponding landmark of the target anatomy on the image data MID. For example, with reference to the example of FIG. 8, the required feature RF can be evaluated in the image MID starting from the distal-most point of the condyle contact surface. Continuing with the illustrated example, the target anatomy TA was originally measured within the image boundary MIB to be 87 mm, and the required feature RF measurement was 92 mm. In FIG. 7, the automated image checking suite AICS automatically measures the required feature RF and determines that the required feature RF extends beyond the image boundary MIB. Hence, the image MID fails to include the required 5 mm of the target anatomy TA.

Additionally, or alternatively, in another technique the automated image checking suite AICS can perform the automated planning pre-check PPC by evaluating the selected implant SI or implant measurements IM relative to the target anatomy TA in the image. In other words, in this technique, the implant parameters themselves are evaluated, and the required feature RF (derived from the implant) need not be obtained or evaluated. Specifically, as shown at step 312, the automated image checking suite AICS can evaluate whether or not any portion of the identified implant SI or implant measurements IM exceeds the medical image boundary MIB. In one example, this can be a check to determine whether the identified implant SI or implant measurement IM is spaced apart from the image boundary by a threshold distance. The threshold distance, for example, can be based on a difference between: the implant measurement IM and the required feature RF; the implant measurement IM and the anatomical measurement AM; or the anatomical measurement AM and the required feature RF Alternatively, the threshold distance can be a pre-defined distance, for example, derived from statistical data. The automated image checking suite AICS can automatically perform this comparison, for example, by registering or correlating the reference coordinates from where the implant measurements IM are taken to the corresponding landmark of the target anatomy TA on the image data MID. For example, with reference to the example of FIG. 8, the implant SI or implant measurements IM can be evaluated in the image MID starting from the distal-most point of the condyle contact surface (shown at landmark AL). In some instances, offset implant distances can be included where the articular surface of the planned implant surface is offset from the native articular surface of the target anatomy TA in the image. Continuing with the illustrated example of FIG. 7, the anatomical measurement AM bound by the image was 89 mm and the implant measurement IM height was 72 mm. Based on these measurements, the automated image checking suite AICS automatically determines that the selected implant SI or implant measurement IM is within the image boundary MIB. Hence, the image MID adequately includes the required image of the target anatomy TA.

Additionally, or alternatively, to compare the implant or implant measurements to the medical image boundary MIB, the selected implant SI can be represented as an implant shape model SSM-I. The automated image checking suite AICS can fit the implant shape model SSM-I to the target anatomy TA in the image MID at a planned location. The automated image checking suite AICS automatically determines whether any portion of the boundaries of the implant shape model SSM-I exceed the image boundary MIB. In the example of FIG. 7, implant shape model SSM-I is captured within the image MID.

After applying one or both techniques described above, the automated planning pre-check PPC (at step 314 of FIG. 6) evaluates whether or not the medical imaging data MID is acceptable. For instance, in the example of FIG. 7, the automated image view pre-check IVPC identified that required feature RF extended beyond the image boundary MIB. Assuming this was the only type of check, the automated planning pre-check PPC would determine at step 314 that the image is unacceptable. The reason for rejection may be characterized as: the image failing to include the required detail of target anatomy TA (e.g., bone) necessary to plan an implant; the image not being large enough to capture the required portion of the target anatomy TA given the implant or not being large enough to capture a region necessary for a planned implant; the target anatomy portion for which an implant will be located is not be captured within the image view; the target anatomy portion for which an implant will be located is clipped; a required portion of the target anatomy which is required for implant planning exceeds the image boundary; and/or a planned implant for the target anatomy will exceed the boundary of the image or is not spaced away from the image boundary by a threshold distance.

If the medical imaging data MID is determined to be unacceptable as a result of the automated planning pre-check PPC, at step 316, can automatically produce a response regarding the unacceptability. In one example, the response is to send a confirmation to the GUI to enable the technician to view that this pre-check has failed or to identify which image slices have failed, or optionally display in the GUI the respective slice and measurements exhibiting the failed boundary comparison. The response can also be to output a message requesting that the patient needs to obtain a larger scan. In another example, the response is for the automated image checking suite AICS to stop processing additional pre-checks, e.g., in an ordered series of checks.

On the other hand, the medical imaging data MID may be determined to be acceptable as a result of this comparison. For instance, in the example of FIG. 7, the shape model implant SSM-I and/or the implant measurement IM are captured inside the image boundary MIB. Assuming these were the only types of checks, then the automated planning pre-check PPC would determine at step 314 that the image is acceptable. The reason for acceptability may be characterized as: the image including the required detail of target anatomy TA (e.g., bone) necessary to plan an implant; the image being large enough to capture the required portion of the target anatomy TA given the implant or being large enough to capture a planned implant; the target anatomy portion for which an implant will be located is captured within the image view; the target anatomy portion for which an implant will be located is not clipped; and/or a planned implant for the target anatomy will not exceed the boundary of the image or is spaced away from the image boundary by a threshold distance.

If acceptable, the automated planning pre-check PPC, at step 318, can automatically produce a response to confirm the acceptability. In one example, the response is to send a confirmation to the GUI to enable the technician to view that this pre-check has passed. In another example, the response is for the automated image checking suite AICS to continue processing additional pre-checks, e.g., in an ordered series of checks. Other responses are contemplated, such as producing no response unless an error was detected.

### 3A. Anatomy/Image/Implant Extrapolation

As described above with reference to section (2A), the automated planning pre-check PPC be similarly equipped with the capability to predictively extrapolate or extend the target anatomy TA, the implant and/or the image MID, shown at step 320 of FIG. 6. If it is determined that a required amount of target anatomy TA required to plan the implant is not captured in the image MID, the automated image checking suite AICS may artificially extend the target anatomy TA and/or implant beyond the original boundary MIB of the image MID. This technique can be incorporated as described above, and the details are not fully repeated for simplicity in description. This extrapolation of the target anatomy, implant and/or image can be performed after the comparison of step 310 (which compares the required feature of the target anatomy to the medical image boundary) or after the comparison of step 312 (which compares the implant measurements to the medical image boundary). After step 310, the target anatomy can be extended using the shape model SM, as described above. After 312, the target anatomy and/or the implant can be extended using the shape model SM, and implant shape model SSM-I, respectively as described above.

This technique is beneficial to save images from rejections and avoid rescanning of images that would otherwise fail the planning pre-check. For example, for primary or revision total knee arthroplasty (TKA), the described extrapolation technique can take current CT scan images and extrapolate the required additional length of the anatomical shape model SM and/or implant shape model SSM-I to account for the desired length of a planned stem, e.g., 100 mm or 200 mm.

### 4. Automated Image Classification Pre-Check

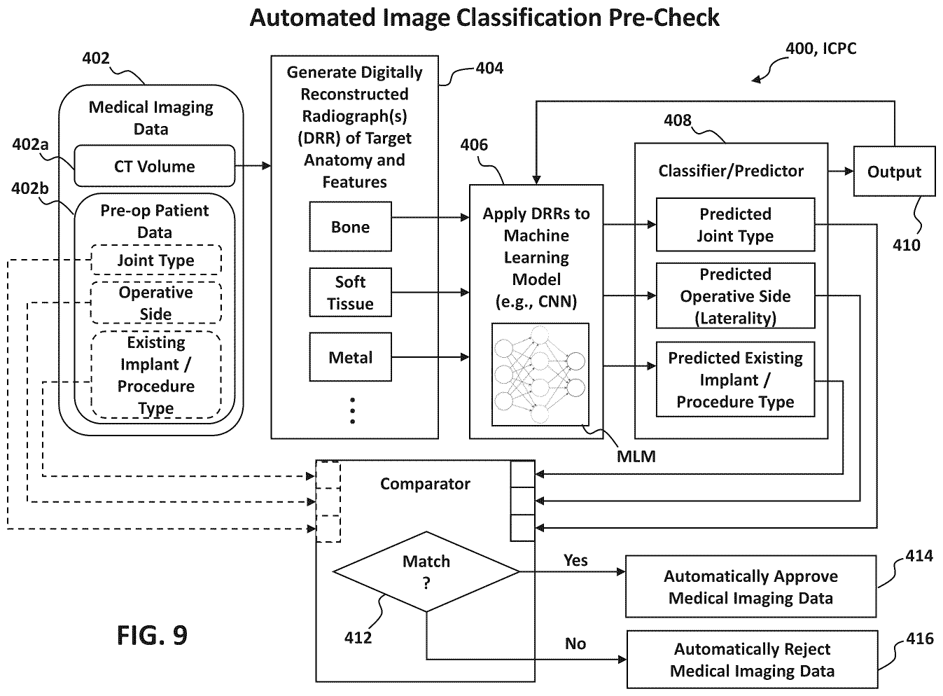

With reference to FIG. 9, the automated image checking suite AICS can perform an automated image classification pre-check ICPC. A primary purpose of the automated image classification pre-check ICPC is to assess whether the imaging data MID represents what the imaging data MID was intended to represent. If the imaging data MID fails to represent what was intended, the imaging data could adversely affect the accuracy and/or completeness of any downstream surgical planning (e.g., segmentation, anatomical model creation, implant planning) or aspects of surgical workflow which rely on surgical planning or medical (e.g., surgical navigation visualization, anatomical registration, etc.). The automated image classification pre-check ICPC efficiently and accurately detects these discrepancies, thereby improving the accuracy and completeness of downstream surgical planning.

One example implementation of the automated image classification pre-check ICPC is illustrated in FIG. 9. Certain features or aspects shown in FIG. 9 may be optional and will be described as such. The automated image classification pre-check ICPC is not strictly limited to exactly the steps described. Certain features of the automated image classification pre-check ICPC may stand-alone or be implemented without the others features. For example, the automated image classification pre-check ICPC may perform classification or predictions, without necessarily performing the described pre-check.

At step 402, the automated image checking suite AICS receives the imaging data MID of the target anatomy TA. For the example of FIG. 9, the imaging data is a volumetric (3D) scan, and more specifically a CT volume scan (402a). However, the imaging data is not limited to a CT scan or a volumetric scan. As described above, the imaging data MID may include any type of image or scan, whether 3D or 2D.

In addition to the CT volume, the imaging data MID further includes data (at 402b), i.e., textual, or numerical data, metadata, and/or any type of information indicative of intended parameters related to the patient, the target anatomy TA, and/or the imaging data MID. The intended parameters are indicative of purported features, aspects, or conclusions related to the target anatomy scanned in the image. The automated image classification pre-check ICPC checks whether these intended parameters are accurate. For example, in FIG. 9, the intended parameters data 402b may include (1) data related to the intended target anatomy that was purportedly scanned in the image, such as the intended type of joint (e.g., knee, hip, shoulder, etc.), or intended parameters of the joint (e.g., joint geometry, kinematics, kinetics, bone density, disease state); (2) intended image or planning data, such as the intended operative side of the patient's anatomy for which the scan was purportedly obtained, or the intended operative side on which the patient's surgery is planned (e.g., left side joint, right side joint, bilateral joint, medial, lateral, etc.), (3) intended procedure, such as whether the procedure is a total joint arthroplasty (e.g., total knee TKA, total hip THA, etc.), a partial joint arthroplasty (e.g., partial knee, partial hip, etc.), a primary surgery or a revision surgery; intended implant for the patient (e.g., total knee implant, partial hip implant, etc.), or the intended manufacturer, model or size of the implant; an intended presence or absence of existing metal, foreign artifact, implant in target anatomy; and/or an intended type of existing metal, foreign artifact, implant in target anatomy.

At step 404, the automated image checking suite AICS optionally converts or generates one or more digitally reconstructed radiographs (DRR) from the imaging data MID (e.g., CT volume). Each of the one or more DRRs is 2D digitally simulated projection of the CT volumetric data. At this step 404, the automated image checking suite AICS generates the one or more DRRs of the target anatomy TA, and optionally, specific features related to the target anatomy TA. For instance, the automated image checking suite AICS can generate one or more DRRs of (1) the bony structure of the target anatomy TA, (2) soft tissue on or surrounding the target anatomy (e.g., including the skin), and/or (3) metal or foreign artifacts located in the target anatomy TA. In one instance, DRRs may be indiscriminately generated for each slice of the volumetric data. Alternatively, the automated image checking suite AICS can utilize the shape modeling techniques described above to preliminary identify these features and generate the DRRs to specifically capture these preliminarily identified features. Other techniques can be used to readily identify bone, soft tissue, or metal, e.g., using material density (Hu) classification, voxel analysis, brightness or contrast comparisons, region of interest classification, etc.

Instead of generating DRRs at step 404, it is contemplated that the automated image checking suite AICS can utilize 2D images (e.g., X-rays or 2D CT slices) to identify specific features related to the target anatomy TA (e.g., bone, soft tissue, metal, etc.). It is also contemplated that 3D volumetric data (such as the original CT volume) may be utilized (without reducing the volume to 2D images or DRRs). The 3D image data can be down sampled. Although the various steps described below are explained in the context of DRRs, it should be understood that the various steps can be implemented using any other type of imaging data format.

At step 406, the automated image checking suite AICS further performs the automated image classification pre-check ICPC by automatically applying the one or more DRRs to a machine learning model MLM. The machine learning model MLM analyzes the one or more DRRs to automatically classify or identify objects or features in the DRRs (such as bone, soft tissue, or metal). The machine learning model MLM can be configured to detect and classify these objects or features using shape or contour recognition, landmark detection, pattern recognition, bounding boxes, or the shape modeling techniques described above. In some instances, the machine learning model MLM may perform image segmentations of the DRRs. The DRRs may be normalized prior to be inputted into the machine learning model MLM to provide a consistent format prior to classification.

In one example, the machine learning model is a neural network, such as a convolutional neural network (CNN), or artificial neural network (ANN). To rapidly process DRRs, the machine learning model may be configured as a 2D lightweight convolutional neural network. The machine learning model may include any number of convolutional layers and connected layers. When the convolutional neural network is utilized, the architecture may include several nodes organized into layers. The DRRs are inputted into an input layer and filtered through various interconnected processing layers. Connections between nodes are assigned weighting values based on the training data and can be adjusted. The output of the neural network is based on the sum of the weighting values. The features within the DRRs can be progressively segmented, classified, or discriminated throughout this process.

The machine learning model MLM can be trained on any medical imaging data, such as medical imaging data related to other patients. For example, the training data may be based on imaging data related to one or more of: patients having characterized target anatomies; patients having procedures on characterized operative sides; patients exhibiting characterized diseased anatomy or healthy anatomy; patients having a characterized type of procedure; post-operative images of patients with characterized implant types, sizes or manufacturers; patients having revision surgery; patients having primary surgery; patients having characterized age, sex, ethnic origin, weight, and/or height; or the like.

The machine learning model MLM may also ingest any of the described pre-operative patient data or intended parameters (provided with the medical imaging data). The machine learning model MLM may do so as a means to accelerate the classification process. For instance, if the medical imaging data identifies that the medical imaging is of a knee joint and the planned procedure is a total knee arthroplasty, the machine learning model MLM may ingest this information to tune the classification algorithm to use training data that is based on medical images of knee joints (e.g., rather than hip joints). Similarly, other patient data, such as age, sex, ethnic origin, weight, and height can be inputted into the machine learning model MLM.

In one example, the convolutional neural network is based on a model-switching architecture that selects a model for segmentation depending on the inputted DRR. For example, if the inputted DRR is of a bone structure, the convolutional neural network may select a model for classifying bone structure. If the inputted DRR is of a soft tissue structure, the convolutional neural network may select a second model for classifying bone structure. If the inputted DRR is of a metal structure, the convolutional neural network may select a third model for classifying metal structures, and so on. This technique of model selection increases classification accuracy and speed while reducing computational load. Additionally, the convolutional neural network may be adaptable to include additional models beyond the models described. For example, if there is a later need to classify existing implant types, a fourth model for classifying implant types can be trained and the convolutional neural network may select this model based on a DRR that exhibits an implant. In one implementation, for any given imaging data set, three separate DRRs may be generated for bone, soft tissue and metal, and the machine learning model MLM simultaneously evaluates the three separate DRRs. The machine learning model MLM may select one model to evaluate one or more DRRs or select one or more models to evaluate several DRRs. Other types of machine learning models can be utilized, such as a deep learning model configured to classify contents of the medical image data or CT volume, including the anatomy types, laterality, treatment or procedure type, existing implants, and the like.

At step 408, after analysis of the medical imaging data (or DRRs) by the machine learning model MLM, the machine learning model MLM outputs predicted parameters. The predicted parameters may include textual, numerical data, metadata, and/or any type of information indicative of predications or classifications related to the patient, the target anatomy TA, and/or the imaging data MID. The predicted parameters are provided to confirm or refute the intended parameters features, aspects, or conclusions related to the target anatomy scanned in the image and/or derived from the patient data (from step 402b). In other words, the automated image classification pre-check ICPC utilizes the predicted parameters to evaluate whether the intended parameters are accurate.

The predicted parameters may include (1) data predicting the target anatomy that was scanned in the image, such as predicting the type of joint (e.g., knee, hip, shoulder, etc.), or predicting parameters of the joint (e.g., joint geometry, kinematics, kinetics, bone density, disease state); (2) predictions of image or planning data, such as predicting the operative side of the patient's anatomy for which the scan was obtained, or predicting the operative side on which the patient's surgery is planned (e.g., left side joint, right side joint, bilateral joint, medial, lateral, etc.), (3) surgical procedure predictions, such as predicting whether the procedure is a total joint arthroplasty (e.g., total knee, total hip, etc.), a partial joint arthroplasty (e.g., partial knee, partial hip, etc.), a primary surgery or a revision surgery; predicting an implant for the patient (e.g., total knee implant, partial hip implant, etc.), predicting the manufacturer, model or size of the implant; predicting presence or absence of existing metal, foreign artifact, implant in target anatomy; and/or predicting the type of existing metal, foreign artifact, implant in target anatomy. For instance, when the medical imaging data is of an anatomical joint, the machine learning model MLM may predict that: the joint is a knee joint, the operative side should be the left-side knee, and there is no existing metal object found in the image of the anatomical joint.

At step 410, the automated image checking suite AICS may optionally output the classification/predictions determined at step 408. This output may be generated prior to performing the comparison to the intended parameters, as will be described in greater detail below. This output 410 may be provided for various purposes. For example, the output 410 may be provided to provide a classification report of the medical imaging data. For instance, the classification report can include any of the predictions related to the target anatomy or procedure that have been described above. The classification report can be provided on the data review screen or GUI for review by a technician. For example, the technician may wish to review whether the imaging data indicates presence or absence of an existing implant for the target anatomy, and if so, the predicted manufacturer or model of the existing implant. The report can be provided for clinical purposes or surgical planning purposes. Additionally, or alternatively, the output 410 may be ingested into the machine learning model MLM at step 406 to provide additional input to and/or improve the training data.

At step 412, the automated image classification pre-check ICPC automatically compares each intended parameter to its corresponding predicted parameter. For example, the intended type of target anatomy is compared to the predicted type of the target anatomy, or the intended operative side of the target anatomy is compared to the predicted operative side of the target anatomy, etc. A comparator module may be implemented to receive and organize the intended parameters from the received medical imaging data 402 and receive and organize the predicted parameters outputted by the machine learning model MLM (from steps 406, 408). The comparator may organize the corresponding intended and predicted parameters in a look-up table. In some cases, the automated image classification pre-check ICPC may know what the intended parameters are beforehand and seek to obtain the specific predicted parameters to confirm/refute the corresponding intended parameters. In other cases, the automated image classification pre-check ICPC may not know what the intended parameters are beforehand. Instead, the automated image classification pre-check ICPC determines what predicted parameters were outputted and checks for whether the corresponding intended parameters was provided.

The comparison may be implemented by determining whether the corresponding intended and predicted parameters are identical, match, correspond, are substantially similar, or otherwise acceptably match. To acceptably match, the corresponding intended and predicted parameters may agree within a threshold tolerance of acceptability. In one example, the threshold tolerance may be greater than 95% confidence score. For instance, the intended parameter may indicate that the scanned anatomy is a left knee joint and the corresponding predicted parameter may indicate a 97% confidence score that the scanned anatomy is a left knee joint. Since the prediction is greater than the threshold, the automated image classification pre-check ICPC may confirm that the target anatomy intended to be scanned was actually scanned. In other examples, to acceptably match, the corresponding intended and predicted parameters must be identical, with zero tolerance. For example, the intended parameter may indicate that the operation is for a right hip. In order for the prediction to acceptably match, the prediction must indicate a 100% confidence score that the operation is for a right hip. The level of confidence or threshold tolerance may be selectively tuned depending on the criticality of the check. In other examples, the machine learning model MLM may be trained with such exceptional accuracy such that level of confidence or threshold tolerance is not regarded. For example, the intended parameter may indicate that the target anatomy comprises no existing implant. If the predicted parameter indicates that the target anatomy comprises no existing implant, then this result may be presumed to be accurate, regardless of the accuracy score. In other examples, if a corresponding intended parameter is missing from the medical imaging data, the comparison may yield an error result, but the predicted parameter may nevertheless be outputted at 410. It is contemplated to perform the comparison between any intended and predicted parameters described above using any other technique or method not specifically described herein.

After applying the comparison described above, the automated image classification pre-check ICPC evaluates whether or not the medical imaging data MID is acceptable. In other words, the automated image classification pre-check ICPC determines, based on the outputted predictions, whether or not the medical imaging data MID represents what it intended to represent.

The medical imaging data MID may be approved in response to a determination that any intended parameter(s) acceptably matches its corresponding predicted parameter(s). Approval may be contingent upon several intended parameters acceptably matching their corresponding predicted parameters. For example, approval may require that the predicted joint type, predicted operative side, and predicted procedure type match the respective intended joint type, intended operative side, and intended procedure type. The automated image classification pre-check ICPC can be configured to select which parameter or grouping of parameters must acceptably match.

If acceptable, the automated image classification pre-check ICPC determines, based on the outputted predictions, that the medical imaging data MID represents what it intended to represent. The automated image classification pre-check ICPC, at step 414, can automatically produce a response to confirm the acceptability. In one example, the response is to send a confirmation to the GUI to enable the technician to view that this pre-check has passed. In another example, the response is for the automated image checking suite AICS to continue processing additional pre-checks, e.g., in an ordered series of checks. Other responses are contemplated, such as producing no response unless a classification discrepancy was detected.

On the other hand, the medical imaging data MID may be rejected in response to a determination that any one or more of the intended parameters fails to acceptably match its corresponding predicted parameter. If rejected, the automated image classification pre-check ICPC determines, based on the outputted predictions, that the medical imaging data MID fails to represents what it intended to represent. If the medical imaging data MID is determined to be unacceptable as a result of the automated image classification pre-check ICPC, at step 416, can automatically produce a response to confirm the unacceptability. In one example, the response is to send an alert or notification to the GUI to enable the technician to view that this pre-check has failed or to identify which intended parameters have failed and why, or optionally display in the GUI the predicted features from the DRRs. The response can also be to output a message requesting that the patient needs to obtain another scan or that the preoperative patient data needs to be reviewed. In another example, the response is for the automated image checking suite AICS to stop processing additional pre-checks, e.g., in an ordered series of checks.

### 5. Automated Motion (Rod) Check

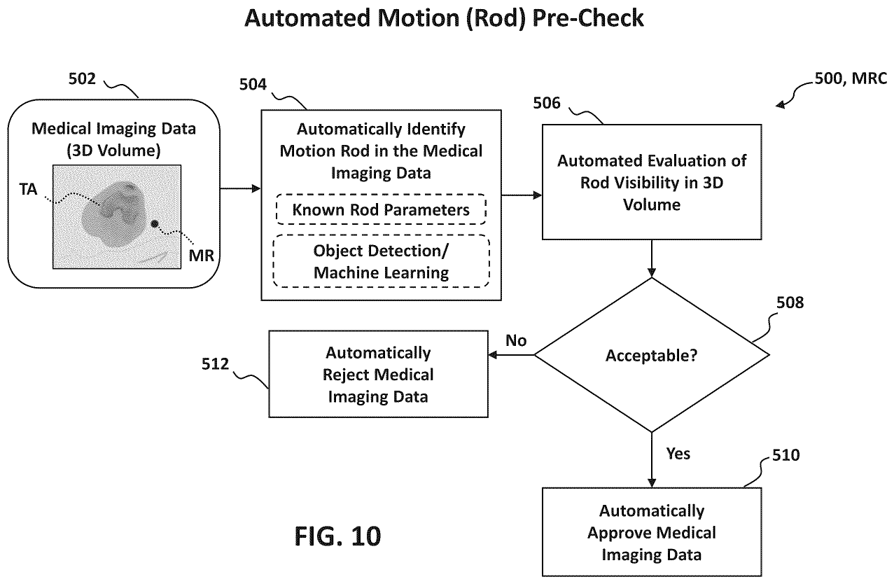

Another pre-check that can be implemented by the automated image checking suite AICS is an automated motion check MRC, as shown in FIG. 1 and as described at method 500 in FIG. 10. The motion check MRC automatically evaluates the medical imaging data MID to determine whether the patient moved during the scanning process. As shown in the example medical image slice of FIG. 10, a motion rod (MR) is present in the image alongside the target anatomy TA. The motion rod MR included as part of a scanning protocol and is a physical bar that is coupled to the patient's anatomy or limb (e.g., leg) during scanning to hold the anatomy still. For example, in preparation for scanning for a knee procedure, the motion rod MR may be strapped to leg of the patient extending from the hip region to the knee region and to the ankle region. The motion rod MR is radiopaque and visible within the scanned images. During scanning, the motion rod MR should remain motionless to ensure the accuracy of the scan. If motion is detected in the rod MR, this will indicate that the patient moved during the scan, which would require a rejection or repeat of the scan.

Accordingly, to implement the automated motion check MRC, the example method 500 of FIG. 10, includes step 502 of receiving the imaging data MID the target anatomy TA and the motion rod MR. For this check, the received imaging data MID is advantageously provided as a 3D CT volume. However, in other implementations, the imaging data MID can be CT slices. At step 504, the automated motion check MRC can automatically identify the motion rod MR in the 3D volume of the medical imaging data MID. This process may involve the automated motion check MRC receiving known parameters of the motion rod MR, such as the rod diameter, radius, length, density, or the like. The automated motion check MRC may additionally or alternatively utilize an object detection algorithm or machine learning algorithm to detect the motion rod MR (with or without known information about the motion rod). The machine learning algorithm may be trained on data sets to automatically distinguish between the motion rod MR and the target anatomy TA or other objects such as existing implants, or the like. Once the motion rod MR is initially identified at step 504, the automated motion check MRC automatically evaluates the entire 3D volume of the medical imaging dataset to determine if the motion rod MR is visible. This step 506 can be performed concurrently as step 504 (i.e., at one time). If slices are utilized, step 506 can include a slice-by-slice evaluation of the motion rod visibility. To determine if the motion rod MR is visible, the automated motion check MRC can evaluate voxels or utilize the object detection or machine learning algorithm to detection presence of the motion rod MR within the 3D volume. Again, this process can involve the known parameters of the motion rod MR, if available. Additionally, the automated motion check MRC can create a shape model (e.g., a cylinder or circle) to fit to the motion rod MR or its cross-section (if slices are used). For volumetric analysis, the automated motion check MRC can check whether the full cylinder is present in the 3D volume and matches the scanned motion rod parameters. The full cylinder indicates that the motion rod MR was stationary and hence the volume was taken while the patient was motionless. If a geometry other than a full cylinder (e.g., such as a partial cylinder, or a blurred geometry) is identified in the volume, the automated motion check MRC automatically identifies that the motion rod MR had moved during scanning and hence the scan was taken while the patient was moving. If slices are used, the automated motion check MRC can check whether a full circle is present in the slice at the location of the motion rod MR. The full circle indicates that the motion rod MR was stationary and hence the slice was taken while the patient was motionless. If a geometry other than a full circle (e.g., such as a partial circle, or a blurred geometry) is identified in a particular slice, the automated motion check MRC automatically identifies that the motion rod MR had moved during scanning and hence the slice was taken while the patient was moving.

At step 508, as a result of evaluating the medical imaging data MID for the motion rod MR, the automated motion check MRC automatically determines whether or not the evaluation results in an acceptable value or falls within a predetermined range or threshold of values. For example, acceptability may mean that the volume or every slice exhibits a completely (100%) visible motion rod MR. Alternatively, acceptability may mean that the volume or every slice exhibits a motion rod MR that is visible above a threshold (e.g., greater than 95% visible). Unacceptable results may mean that the volume or at least one slice fails to exhibit a completely visible motion rod MR. Alternatively, unacceptable results may mean that the volume or at least one slice fails to exhibit a motion rod MR that is visible above a threshold. The thresholds for acceptability and unacceptability may be the same or different.

If the evaluation is acceptable, the automated motion check MRC, at step 510, can automatically produce a response to confirm the acceptability. In one example, the response is to send a confirmation to the GUI to enable the technician to view that this pre-check has passed or that the scan was properly obtained without patient motion. In another example, the response is for the automated image checking suite AICS to continue processing additional pre-checks, e.g., in an ordered series of checks. Other responses are contemplated, such as producing no response unless an error was detected.

If the evaluation is unacceptable, the automated motion check MRC, at step 512, can automatically produce a response regarding the unacceptability. In one example, the response is to send an alert or notification to the GUI to enable the technician to view that this pre-check has failed or to identify which slices failed, if applicable. In another example, the response is for the automated image checking suite AICS to stop processing additional pre-checks, e.g., in an ordered series of checks. The response can also be to output a message requesting a new scan be taken with the patient being motionless.

### 6. Automated Volume Classifier and Laterality Pre-Check

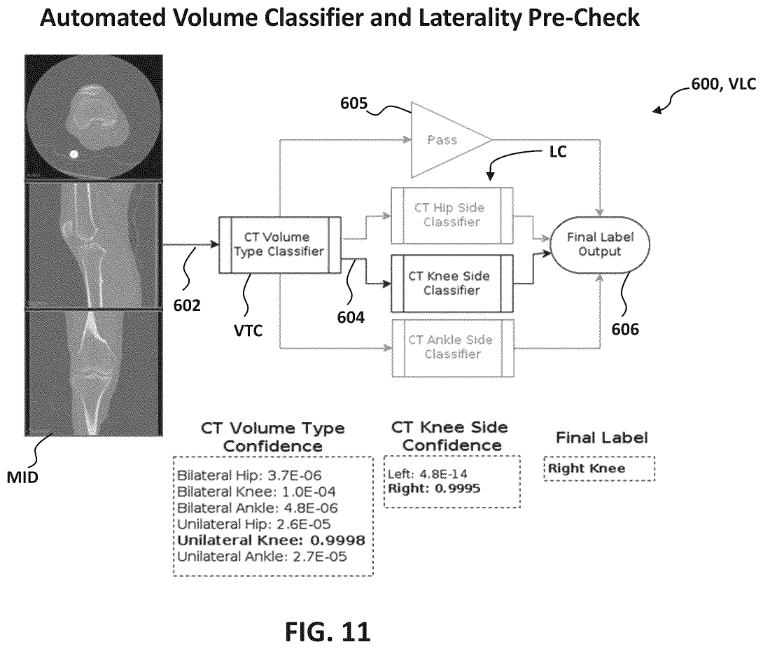

Yet another module that can be implemented by the automated image checking suite AICS is an automated volume classifier and laterality pre-check or classifier VLC, as shown in FIG. 1 and as described with reference to FIG. 11 and method 600. The VLC pre-check can be included in addition to, or utilized as a sub-feature of, any of the described pre-checks.

The VLC pre-check includes a pipeline that can identify the volume type contained within a given 3D image volume of medical imaging data MID. The VLC pre-check can also identify laterality of the anatomy for volumes that are of unilateral type. Based on this pipeline, the VLC pre-check outputs a final label identifying the volume type and laterality.

With reference to FIG. 11, the medical imaging data MID inputted into the VLC pre-check at step 602. The medical imaging data MID can be a 3D volume, e.g., a 3D CT volume from a DICOM series. The medical imaging data MID can be of any type of anatomy, including but not limited to: knee, ankle, hip, shoulder, spine, etc. In one example, the CT volume is unclassified prior to be inputted into the VLC pre-check. In other words, the CT volume may comprise no information to identify the type of anatomical volume imaged, nor the laterality of the anatomical volume imaged.

The medical imaging data MID can be inputted into a volume type classifier VTC, implemented by a first part of the VLC pre-check pipeline. The volume type classifier VTC can be implemented as a deep learning model that calculates the likelihood of a 3D volume being one of a plurality of volume types. The volume types include, but are not limited to: unilateral hip, unilateral knee, unilateral ankle, bilateral hip, bilateral knee, or bilateral ankle. The most likely of these is taken to be the output of the volume type classifier. For example, as shown in FIG. 11, the output of the volume type classifier VTC is an identification that the medical imaging data MID contains a unilateral knee. Confidence values may be recorded or presented on the GUI or a report. Here, the volume type classifier VTC produces a confidence score of 0.9998 for unilateral knee. In one example, if the confidence score is above a threshold, e.g., greater than 95%, 98%, or 99%, the volume type classifier VTC can output the result, at 604, to the next step of the VLC pre-check pipeline, i.e., the side or laterality classifier LC. Additionally, or alternatively, the highest confidence score is taken as the output of the volume type classifier VTC.

In one implementation, the volume type classifier VTC can output the result to the laterality classifier LC only in response to classifying the volume as a “unilateral” bone structure (hip, knee ankle, etc.) because a unilateral bone structure exhibits a higher susceptibility of confusion of laterality as compared to bilateral due to the absence of the respective left or right side of the anatomy, which provides a comparison for reference. In such cases, if the volume type classifier VTC classifies the volume as a “bilateral” bone structure, the volume type classifier VTC can bypass the laterality classifier LC, at 605, and output the result directly to the final label output at 606. Of course, it is contemplated the volume type classifier VTC can output the result to the laterality classifier LC in response to classifying the volume as a “bilateral” bone structure as well.

In one example, the deep learning model utilized for the volume type classifier VTC is a DenseNet-264, densely connected convolutional network architecture. Training images were rescaled to maintain aspect of the original CT image and the voxel intensities were clipped to a specific Hounsfield unit range. During each training epoch, images were randomly sampled according to their labels (“left hip” or “right hip”) to ensure the model was trained on an approximately equal number of each. Intensity scaling was performed on the voxels to compensate for variability in CT scanners and CT scanner calibration. Affine transforms (shear and rotation) were applied to the training images to compensate for changes in patient position in the scanner, and scaling was included to compensate for variability in the height and weight of patients. Other types of machine learning models can be utilized to classify contents of the medical image data or CT volume, such as a convolutional neural network, a random forest classifier, a decision tree classifier, a K-Nearest neighbor classifier, a Naive Bayes classifier, a support vector machine, or the like.

To implement the laterality classifier LC, the VLC pre-check can include a CT hip side classifier that can be implemented as a deep learning model that calculates the likelihood of a 3D volume being a left or right hip. The most likely of these is taken to be the output of the classifier. The VLC pre-check can include a CT knee side classifier that can be implemented as a deep learning model that calculates the likelihood of a 3D volume being a left or right knee. The most likely of these is taken to be the output of the classifier. The VLC pre-check can include a CT ankle side classifier that can be implemented as a deep learning model that calculates the likelihood of a 3D volume being a left or right ankle. The most likely of these is taken to be the output of the classifier.

These laterality classifiers LC can be implemented separately or combined into one single classifier. When the laterality classifiers LC are separated, as shown in FIG. 11, the output from the volume type classifier VTC will input directly into the respective the laterality classifier LC related to the classified volume. For example, in FIG. 11, the output of the volume type classifier VTC is a unilateral knee. Hence, the VLC pre-check has successfully identified the type of anatomy in the imaging but has not yet identified the laterality of this knee. Accordingly, the output of the volume type classifier VTC can be inputted directly into the CT knee side classifier, which is specifically trained to detect the laterality of the knee in an imaging volume. This design choice of splitting laterality classifiers by bone type can decrease computation time and enable large quantities of imaging data to be processed.

As shown in FIG. 11, the output of the laterality classifier LC is an identification of the laterality of the anatomy from the medical imaging data MID. Confidence values may be recorded or presented on the GUI or a report. In the example shown, the laterality classifier LC produces a confidence score of 0.9995 indicating that the knee is a right knee. In one example, if the confidence score is above a threshold, e.g., greater than 95%, 98%, or 99%, the laterality classifier LC can output the result, at 606, to a final label output. Additionally, or alternatively, the highest confidence score is taken as the output of the laterality classifier LC. For example, if the left knee confidence was 0.05 and the right knee confidence was 0.95, the laterality classifier LC can take the right knee as the proper laterality identification for final label output, at 606. The final label output 606, e.g., “right knee” can be provided on a report or GUI, such as those that will be shown and described herein.

In one example, the deep learning model utilized for any one or more of the laterality classifiers LC is a DenseNet-121, densely connected convolutional network architecture. Training images were rescaled to maintain aspect of the original CT image and the voxel intensities were clipped to a specific Hounsfield unit range. During each training epoch, images were randomly sampled according to their labels (“left hip” or “right hip”) to ensure the model was trained on an approximately equal number of each. Intensity scaling was performed on the voxels to compensate for variability in CT scanners and CT scanner calibration. Affine transforms (shear and rotation) were applied to the training images to compensate for changes in patient position in the scanner, and scaling was included to compensate for variability in the height and weight of patients. Other types of machine learning models can be utilized to classify laterality of the medical image data or CT volume, such as a convolutional neural network, a random forest classifier, a decision tree classifier, a K-Nearest neighbor classifier, a Naive Bayes classifier, a support vector machine, or the like.

The VLC pre-check can use the label output 606 to perform an automated check to determine whether the medical imaging data MID is acceptable or not. This check can be automatically executed in a number of ways. For example, the VLC pre-check can automatically assess the confidence score of the output type and laterality to determine whether the confidence score is above a threshold. If either value is below the threshold, the check can output a failure result. If both values are above the threshold, the check can output a pass result. Additionally, outputs from other pre-checks from the AICS can be utilized to feed into the confidence score from the VLC pre-check. In some instances, the output of the volume type and laterality can be presented for manual review by a technician to review.

### B. Example Planning Workflows

Referring back to FIG. 1, if the automated image checking suite AICS outputs a successful approval or the medical imaging data MID after executing any one or more of the described pre-checks, the treatment planning request, including the approved medical imaging data MID can be passed downstream for other procedures or processes involving surgical planning. For example, at SG, an automated or manual segmentation procedure may commence based on the approved medical imaging data MID. At MC, an anatomical or virtual model may be created based on the segmented image data or the approved medical imaging data MID. At SP, surgical planning can be implemented with respect to the approved medical imaging data MID or the anatomical or virtual model. Surgical planning may be implant planning and may be based on any of the predicted parameters described above. After surgical planning is completed, the treatment planning request may be returned to the appropriate requestor, at RPL. After the planning request is returned, the medical imaging data MID that was approved using the techniques described herein can further be relied on for surgical workflow purposes.

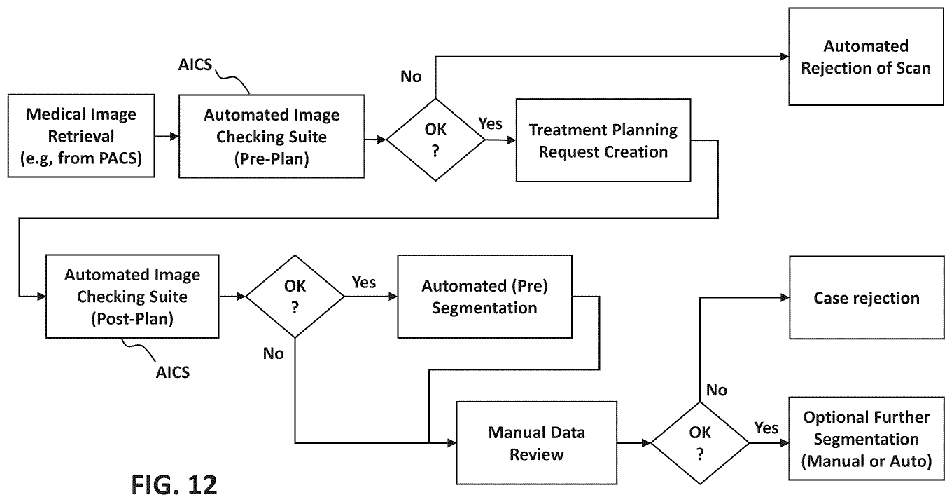

In FIG. 12, another example workflow is provided involving the automated image checking suite AICS. In this example, the treatment planning request is generated after the medical imaging data MID is initially processed by the automated image checking suite AICS. The medical image is initially inputted to the automated image checking suite AICS, e.g., from the PACS server. This input can be automatically performed and uploaded, for example, to a server that runs the automated image checking suite AICS. The automated image checking suite AICS then performs any one or more of the automated image pre-checks that will be described herein. If the result of the automated image pre-checking is unsuccessful, e.g., one or more of the checks has failed, the scan will be automatically rejected by the AICS. Feedback can be automatically provided to appropriate representative or technician, as will be described below. The feedback can be provided in the GUI and can include detailed reports summarizing the reasons for rejection, recommendations for how to correct the scan, instructions requesting a re-scan, etc. If the result of the automated image pre-checking is successful, the workflow continues to creation of the treatment planning request. The appropriate representative or technician can then create the treatment planning request knowing that the medical imaging has initially passed the auto pre-checks, and hence, is initially suitable to use for planning purposes.

With continued reference to FIG. 12, after the treatment plan is created, the plan, including the medical image data, can be inputted once again to the automated image checking suite AICS. The automated image checking suite AICS again performs a “post-plan” processing of any one or more of the automated image pre-checks that will be described herein. Here, the checks be the same or different from than the checks that were performed prior to creation of the treatment planning request. If the result of the (post plan) automated image pre-checking is unsuccessful, e.g., one or more of the checks has failed, then a representative or technician from the segmentation team can be automatically informed of the rejection and can perform a data review of the output. Data review may include reviewing a report of the rejection, identifying potential issues, and performing corrective action. Feedback can similarly be provided to appropriate segmentation representative or technician through the GUI to facilitate data review. On the other hand, if the result of the (post plan) automated image pre-checking is successful, the workflow continues to automatic pre-segmentation of the target anatomy TA in the medical imaging data. The automatic pre-segmentation can perform a coarse segmentation of the target anatomy TA that will be later refined by the technician. Alternatively, the automatic pre-segmentation can perform a full segmentation that will be later reviewed by the technician. The automatic pre-segmentation can be robustly executed at this step based on the confidence that the medical imaging has twice passed iterations of the auto pre-checks, and hence, is suitable to use for segmentation purposes. Pursuant to automated pre-segmentation, the representative or technician from the segmentation team can be informed of the segmentation output and can perform a data review of the output. Data review may include reviewing the segmentation accuracy, output of the pre-checks, identifying potential issues, and performing corrective action. If the data review process is successful, a refinement of the segmentation output can be performed (automatically or manually by the segmentation technician). If the data review process yields a negative result, the case can be rejected with optional case notes indicating the reasons of rejection.

### C. Graphical User Interface Outputs

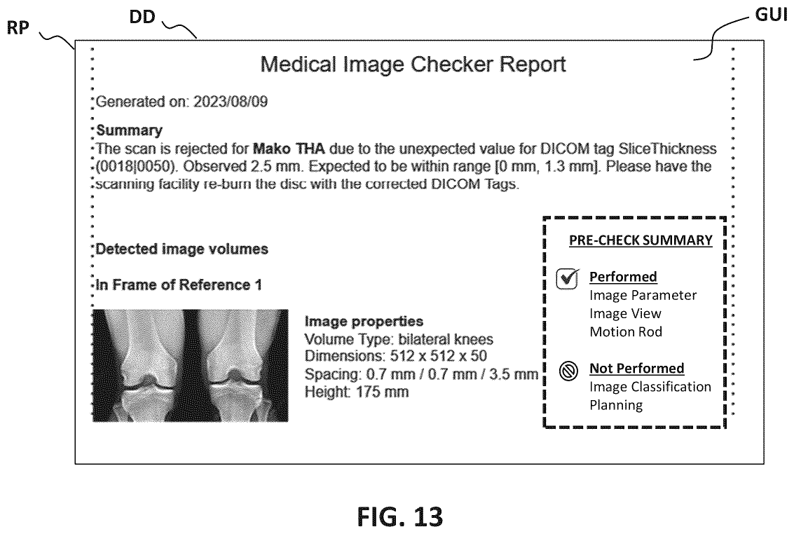

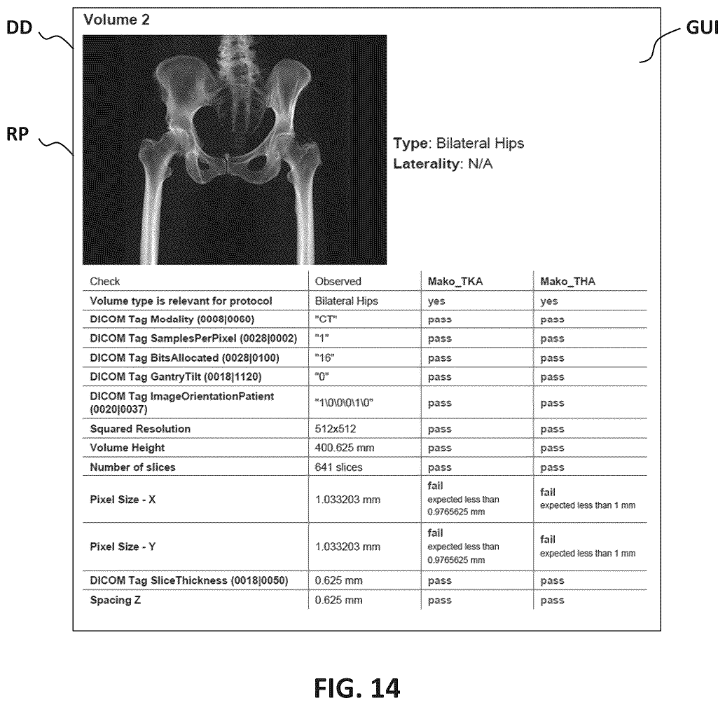

Referring to FIGS. 13-15, additional features/outputs/interfaces are provided for the GUI that can be utilized with the automated image checking suite AICS. In FIGS. 13 and 14, example screens of the GUI are provided which illustrate reports RP that can be automatically generated by the automated image checking suite AICS. Such reports can be generated whether the outcome of any one or more of the pre-checks is acceptable or unacceptable. The report RP can be automatically as a result of any one or more of steps 110, 112, 210, 212, 316, 318, 414, 416, 510, 512, 604, 606 described above for the various pre-checks. The report RP can also be automatically generated in response to the pre-plan or post-plan review by the AICS as described in workflows of FIGS. 1 and 12, for example. The report RP can provide various types of information to assist a technician performing a review of the images, for example, in preparation for segmentation or planning purposes. The information on the report RP can include for instance any of the following: the data the report was generated, a summary of the report, including the specific reasons for the rejection as compared with the acceptable thresholds (if applicable); a recommendation on how to correct the scan; a recommendation to re-scan; an image or representation of particular slices of the medical imaging data MID, such as those slices that may have caused the rejection; properties of the image or target anatomy, such as volume type, dimensions, spacing, height; the name of the patient; the DOB of the patient; the name of the requesting surgeon; the intended type of procedure (e.g., THA, TKA); a summary of what checks were or were not executed by the AICS and whether such checks passed or failed, and why; a confidence or accuracy score for any check; a recommendation for modification to the segmentation process or planning process, and the like.

Failures in the report may be explained, for example, that the scan is rejected due to the anatomy region not being included in the image. A recommendation may be provided in the report to correct the issue, such as to have the scanning facility re-burn the disc with all regions included. Additionally, the report may include predictions or suggestions to identify the root cause of the issue, such as, the observed pixel size of the anatomy being less than an expected pixel size; an observed slice thickness not being within an expected range; a slicing interval not being compliant with protocol (e.g., observed slicing interval was 2.5 mm but the slice interval must be a maximum of less than 1.1 mm with no gaps or overlap). These parameters may be checked, for example, by the automated image parameter check IPPC, as shown and described with reference to FIG. 3.

Additional information provided on the report, for example, as shown in FIG. 14, include imaging acquisition parameters or DICOM tag information, such as modality (e.g., CT), samples per pixel (e.g., 1), bits allocated (e.g., 16), gantry tilt (e.g., 0), image orientation of the patient (e.g., 1\0\0\0\1\0), etc. The report can provide squared resolution of the image, volume height, number of slices, pixel size in X and Y, slice thickness, and Z-spacing. These parameters may be checked, for example, by the automated image parameter check IPPC, as shown and described with reference to FIG. 3.

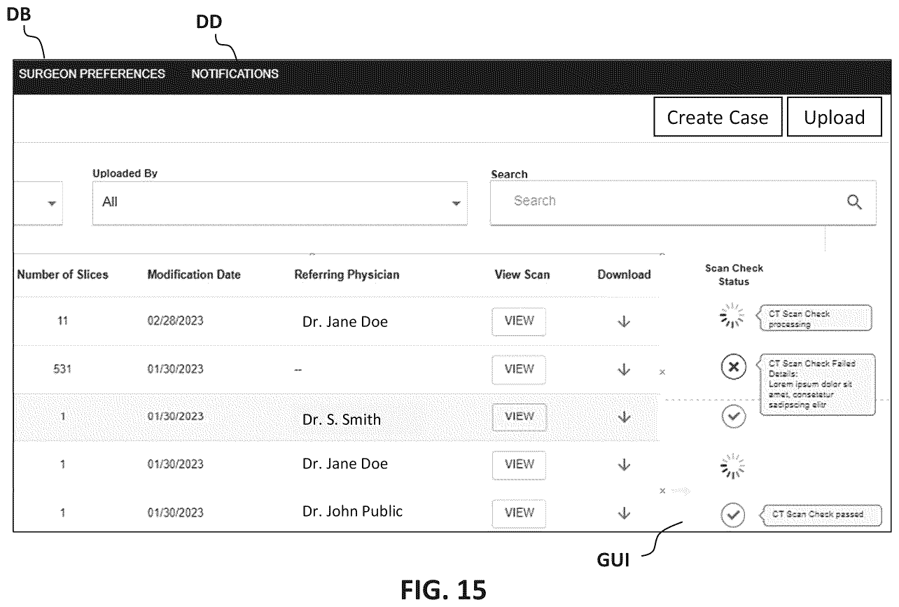

Referring to FIG. 15, a dashboard DB can be implemented by the GUI to provide organization and management of the patient case files. The dashboard DB can be dynamically updated with various information about the case files, such as the patient's name, number of slices of medical imaging data MID, the surgeon's name, and the like. A button can be provided to provide immediate access, download or retrieval of the medical imaging data MID for view. Advantageously, the dashboard DB is provided with a status check. The status check is dynamically updated based on the automated image checking suite's AICS on-going evaluation of medical imaging data for the various case files. The status check can include icons that are graphically presented on the GUI and updated dynamically based on status changes. The icons can indicate, for example, that the check or checks is/are processing, completed, have failed, or have passed. The icons can be checkmarks (if successful), X-marks (if unsuccessful), or can be loading graphics (to indicate the check is in process). The AICS can be integrated with the dashboard, e.g., through an API, or otherwise, to provide the dynamic status updates for each case file. Additionally, the AICS can automatically load relevant notes about the status, such as that the check or checks is/are processing, completed, have failed, or have passed, as well as any information that could be provided in the report RP described above, such as: the specific reasons for the rejection as compared with the acceptable thresholds (if applicable); a recommendation on how to correct the scan; a recommendation to re-scan; an image or representation of particular slices of the medical imaging data MID, such as those slices that may have caused the rejection; a summary of what checks were or were not executed by the AICS; a confidence or accuracy score for any check; a recommendation for modification to the segmentation process or planning process, and the like. Additionally, the dashboard DB can provide direct links for the respective case file to access/download/view any report RP, such as that described above. In turn, between the report RP and the dashboard, the system provides a level of imaging checking management that is exceptionally user-friendly and that provides immediate insight into the automated imaging checking activity performed by the AICS.

### D. Example Technical Solutions and Advantages

As evident from the detailed description and figures, the pre-checks provided by the automated image checking suite AICS provide significant advantages and technical solutions by automatically, rapidly, and accurately identifying deficiencies or errors in the imaging data MID. In doing so, the automated image checking suite AICS can automatically determine whether or not the imaging data MID is suitable for downstream post-processing and/or surgical planning (e.g., segmentation, anatomical model creation, implant planning) or suitable for aspects of surgical workflow which rely on surgical planning or medical imaging (e.g., surgical navigation visualization, anatomical registration, etc.). The automated image checking suite AICS can process the described pre-checks almost immediately, within seconds, thereby alleviating the time-consuming burden of having a technical team manually review the imaging data MID to identify potential errors. In turn, the automated image checking suite AICS can substantially reduce the labor cost and human error involved with manual review of the imaging data. The automated image checking suite AICS can advantageously be performed before and/or after treatment request planning creation, thereby providing additional confidences in before planning and segmentation. By performing the automated checks earlier in the workflow (e.g., before the treatment planning request or segmentation), the amount of manual checking by technicians is greatly reduced thereby freeing up time for the technicians to process significantly more cases. Additionally, the automated checks provide feedback much earlier in the process, thereby avoiding unnecessary delay and downstream waste of resources. The automated image checking suite AICS can perform automated rejections of scans and provide automated reports and summaries to quickly assist technicians in processing large amounts of patient data, thereby providing significant improvements in reducing human error and labor costs. Furthermore, by providing automated feedback, e.g., through reports of the dashboard, the AICS and GUI can provide aggregated summaries for educating scanning technicians to potentially reduce the image rejection rate in the future. Additionally, the automated image checking suite AICS can process and classify unclassified images, thereby not relying on pre-classifications performed by technicians or radiologists, which may be prone to human error. Furthermore, classifiers may be specifically trained using imaging datasets that are specific for certain types of anatomies (e.g., knee, hip, ankles), thereby providing a fast, robust means of processing numerous medical images. Other advantages not specifically described herein are readily understood by those skilled in the art.

Several embodiments have been discussed in the foregoing description. However, the embodiments discussed herein are not intended to be exhaustive or limit the invention to any particular form. The terminology which has been used is intended to be in the nature of words of description rather than of limitation. Many modifications and variations are possible in light of the above teachings and the invention may be practiced otherwise than as specifically described.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

What is claimed is:

1. An automated image checking suite configured to automatically evaluate whether a medical imaging data of a target anatomy is acceptable to facilitate surgical planning for the target anatomy, the automated image checking suite comprising a non-transitory computer readable medium including instructions, which when executed by one or more processors, are configured to:
execute automated checks to determine whether:
the medical imaging data was scanned according to acceptable configuration settings;
the target anatomy in the medical imaging data is acceptably captured within a boundary of the medical imaging data;
the medical imaging data exhibits a motion rod that is visible above a threshold level of visibility; and
the medical imaging data acceptably shows an intended type of target anatomy and an intended laterality of the target anatomy; and
automatically reject the medical imaging data as being unacceptable to facilitate surgical planning in response to a determination that any one or more of the automated checks produces an unacceptable result.

2. The automated image checking suite of claim 1, wherein to determine whether the medical imaging data was scanned according to the acceptable configuration settings, the instructions, when executed by the one or more processors, are configured to:
automatically obtain one or more configuration settings defining how the medical imaging data was scanned by an imaging device;
automatically compare the one or more configuration settings to one or more acceptable configuration settings; and
automatically reject the medical imaging data as being unacceptable to facilitate surgical planning in response to a determination that the one or more configuration settings fail to correspond to the one or more acceptable configuration settings.

3. The automated image checking suite of claim 1, wherein to determine whether the target anatomy in the medical imaging data is acceptably captured within the boundary of the medical imaging data, the instructions, when executed by the one or more processors, are configured to:
automatically identify and fit a shape model to the target anatomy in the medical imaging data;
automatically compare a feature of the shape model to the boundary of the medical imaging data; and
automatically reject the medical imaging data as being unacceptable to facilitate surgical planning in response to a determination that the feature of the shape model exceeds the boundary of the medical imaging data.

4. The automated image checking suite of claim 1, wherein to determine whether the target anatomy in the medical imaging data is acceptably captured within the boundary of the medical imaging data, the instructions, when executed by the one or more processors, are configured to:
automatically compare the medical imaging data to a statistical population of medical imaging datum including other anatomies comparable to the target anatomy to identify an anatomical landmark of the target anatomy that is required to be visible in the medical imaging data;
automatically evaluate the medical imaging data to determine whether the anatomical landmark is visible in the medical imaging data; and
automatically reject the medical imaging data as being unacceptable to facilitate surgical planning in response to a determination that the anatomical landmark fails to be visible in the medical imaging data.

5. The automated image checking suite of claim 1, wherein to determine whether the medical imaging data exhibits the motion rod that is visible above the threshold level of visibility, the instructions, when executed by the one or more processors, are configured to:
automatically identify the motion rod in a volume of the medical imaging data;
automatically evaluate the volume of the medical imaging data to determine if the volume of the medical imaging data acceptably exhibits the motion rod; and
automatically reject the medical imaging data as being unacceptable to facilitate surgical planning in response to a determination that the volume of the medical imaging data fails to acceptably exhibit the motion rod.

6. The automated image checking suite of claim 5, wherein to automatically identify the motion rod, the instructions, when executed by the one or more processors, are configured to:
implement an object detection algorithm or machine learning model to automatically identify the motion rod in the volume and distinguish the motion rod from other features exhibited in the volume.

7. The automated image checking suite of claim 6, wherein to automatically evaluate the volume of the medical imaging data to determine if the volume acceptably exhibits the motion rod, the instructions, when executed by the one or more processors, are configured to:
implement the object detection algorithm or the machine learning model to automatically determine whether the motion rod exhibits a full cylinder in the volume.

8. The automated image checking suite of claim 1, wherein to determine whether the medical imaging data acceptably shows the intended type of the target anatomy and the intended laterality of the target anatomy, the instructions, when executed by the one or more processors, are configured to:
receive the medical imaging data as an input, wherein a type and a laterality of the target anatomy are unclassified in the medical imaging data at a time of input;
automatically classify the type of the target anatomy in the medical imaging data using a first machine learning model;
utilize the classified type of the target anatomy to select a second machine learning model specifically trained to classify the laterality of the classified type of the target anatomy; and
automatically classify the laterality of the target anatomy in the medical imaging data using the second machine learning model.

9. The automated image checking suite of claim 8, wherein the instructions, when executed by the one or more processors, are configured to:
generate a confidence score that indicates classification accuracy of the type of the target anatomy in the medical imaging data; and
automatically reject the medical imaging data as being unacceptable to facilitate surgical planning in response to a determination that confidence score fails to meet an acceptable threshold.

10. The automated image checking suite of claim 8, wherein the instructions, when executed by the one or more processors, are configured to:
generate a confidence score that indicates classification accuracy of the laterality of the target anatomy in the medical imaging data; and
automatically reject the medical imaging data as being unacceptable to facilitate surgical planning in response to a determination that confidence score fails to meet an acceptable threshold.

11. The automated image checking suite of claim 1, wherein the instructions, when executed by the one or more processors, are configured to:
execute automated checks to determine that the target anatomy in the medical imaging data is not acceptably captured within the boundary of the medical imaging data;
identify and fit a shape model to the target anatomy in the medical imaging data;
compare the shape model to the boundary of the medical imaging data;
determine that a portion of the shape model exceeds the boundary of the medical imaging data; and
modify the medical imaging data to capture the portion of the shape model that exceeds the boundary of the medical imaging data.

12. A computer-implemented method for automatically evaluating whether a medical imaging data of a target anatomy is acceptable to facilitate surgical planning for the target anatomy, the computer-implemented method comprising:
executing automated checks for determining whether:
the medical imaging data was scanned according to acceptable configuration settings;
the target anatomy in the medical imaging data is acceptably captured within a boundary of the medical imaging data;
the medical imaging data exhibits a motion rod that is visible above a threshold level of visibility; and
the medical imaging data acceptably shows an intended type of target anatomy and an intended laterality of the target anatomy; and
automatically rejecting the medical imaging data as being unacceptable to facilitate surgical planning in response to determining that any one or more of the automated checks produces an unacceptable result.

13. The computer-implemented method of claim 12, wherein determining whether the medical imaging data was scanned according to the acceptable configuration settings comprises:
automatically obtaining one or more configuration settings defining how the medical imaging data was scanned by an imaging device;
automatically comparing the one or more configuration settings to one or more acceptable configuration settings; and
automatically rejecting the medical imaging data as being unacceptable to facilitate surgical planning in response to determining that the one or more configuration settings fail to correspond to the one or more acceptable configuration settings.

14. The computer-implemented method of claim 12, wherein determining whether the target anatomy in the medical imaging data is acceptably captured within the boundary of the medical imaging data comprises:
automatically identifying and fitting a shape model to the target anatomy in the medical imaging data;
automatically comparing a feature of the shape model to the boundary of the medical imaging data; and
automatically rejecting the medical imaging data as being unacceptable to facilitate surgical planning in response to determining that the feature of the shape model exceeds the boundary of the medical imaging data.

15. The computer-implemented method of claim 12, wherein determining whether the target anatomy in the medical imaging data is acceptably captured within the boundary of the medical imaging data comprises:
automatically comparing the medical imaging data to a statistical population of medical imaging datum including other anatomies comparable to the target anatomy for identifying an anatomical landmark of the target anatomy that is required to be visible in the medical imaging data;
automatically evaluating the medical imaging data for determining whether the anatomical landmark is visible in the medical imaging data; and
automatically rejecting the medical imaging data as being unacceptable to facilitate surgical planning in response to determining that the anatomical landmark fails to be visible in the medical imaging data.

16. The computer-implemented method of claim 12, wherein determining whether the medical imaging data exhibits the motion rod that is visible above the threshold level of visibility comprises:
automatically identifying the motion rod in a volume of the medical imaging data;
automatically evaluating the volume of the medical imaging data for determining if the volume of the medical imaging data acceptably exhibits the motion rod; and
automatically rejecting the medical imaging data as being unacceptable to facilitate surgical planning in response to determining that the volume of the medical imaging data fails to acceptably exhibit the motion rod.

17. The computer-implemented method of claim 16, wherein automatically identifying the motion rod comprises:
implementing an object detection algorithm or machine learning model for automatically identifying the motion rod in the volume and distinguishing the motion rod from other features exhibited in the volume.

18. The computer-implemented method of claim 12, wherein determining whether the medical imaging data acceptably shows the intended type of the target anatomy and the intended laterality of the target anatomy comprises:
receiving the medical imaging data as an input, wherein a type and a laterality of the target anatomy are unclassified in the medical imaging data at a time of input;
automatically classifying the type of the target anatomy in the medical imaging data using a first machine learning model;
utilizing the classified type of the target anatomy for selecting a second machine learning model specifically trained for classifying the laterality of the classified type of the target anatomy; and
automatically classifying the laterality of the target anatomy in the medical imaging data using the second machine learning model.

19. The computer-implemented method of claim 18, comprising:
generating a first confidence score that indicates classification accuracy of the type of the target anatomy in the medical imaging data;
generating a second confidence score that indicates classification accuracy of the laterality of the target anatomy in the medical imaging data; and
automatically rejecting the medical imaging data as being unacceptable to facilitate surgical planning in response to determining that one or both of the first confidence score and the second confidence score fail to meet an acceptable threshold.

20. The computer-implemented method of claim 12, comprising:
executing the automated checks for determining that the target anatomy in the medical imaging data is not acceptably captured within the boundary of the medical imaging data;
identifying and fitting a shape model to the target anatomy in the medical imaging data;
comparing the shape model to the boundary of the medical imaging data;
determining that a portion of the shape model exceeds the boundary of the medical imaging data; and
modifying the medical imaging data for capturing the portion of the shape model that exceeds the boundary of the medical imaging data.
